(19) United States
(12) Patent Application Publication
(10) Pub. No.: US 2006/0035958 A1
(43) Pub. Date: Feb. 16, 2006

(54) Method of treating diabetes and related conditions

(76) Inventors: Joseph Duffy, Cranford, NJ (US); Elizabeth Louise Campbell, North Brunswick, NJ (US); Sajjad A Qureshi, Westfield, NJ (US); Bei B Zhang, Edison, NJ (US); James R Tata, Westfield, NJ (US); Zenon Konteatis, Chatham Township, NJ (US); Rui Liang, East Brunswick, NJ (US)

Correspondence Address: MERCK AND CO., INC, P O BOX 2000, RAHWAY, NJ, 07065-0907, US

(21) Appl. No.: 10/527,652

(22) PCT Filed: Sep. 8, 2003
(86) PCT No.: PCT/US03/28044

§ 371 Date: Mar. 14, 2005

Related U.S. Application Data
(60) Provisional application No. 60/410,254, filed on Sep. 12, 2002.

(51) Int. Cl. A61K 31/381

(52) U.S. Cl. 514/430; 514/443

(57) ABSTRACT

The present invention addresses the use of substituted thiophene derivatives, as well as compositions containing such compounds for treating type 2 diabetes mellitus. The compounds in the present invention are glucagon antagonists. The compounds block the action of glucagon at its receptor and thereby decrease the levels of plasma glucose providing a treatment of diabetes.

### BACKGROUND OF THE INVENTION

The present invention relates to a method of treating type 2 diabetes mellitus and related conditions using substituted thiophene derivatives as well as compositions containing such compounds.

Diabetes refers to a disease process derived from multiple causative factors and is characterized by elevated levels of plasma glucose (hyperglycemia) in the fasting state or following glucose administration during an oral glucose tolerance test. Frank diabetes mellitus (e.g., a blood glucose level ≧126 mg/dL in a fasting state) is associated with increased and premature cardiovascular morbidity and mortality, and is related directly and indirectly to various metabolic conditions, including alterations of lipid, lipoprotein and apolipoprotein metabolism.

Patients with non-insulin dependent diabetes mellitus (type 2 diabetes mellitus), approximately 95% of patients with diabetes mellitus, frequently display elevated levels of serum lipids, such as cholesterol and triglycerides, and have poor blood-lipid profiles, with high levels of LDL-cholesterol and low levels of HDL-cholesterol. Those suffering from Type 2 diabetes mellitus are thus at an increased risk of developing macrovascular and microvascular complications, including coronary heart disease, stroke, peripheral vascular disease, hypertension (for example, blood pressure ≦130/80 mmHg in a resting state), nephropathy, neuropathy and retinopathy.

Patients having type 2 diabetes mellitus characteristically exhibit elevated plasma insulin levels compared with nondiabetic patients; these patients have developed a resistance to insulin stimulation of glucose and lipid metabolism in the main insulin-sensitive tissues (muscle, liver and adipose tissues). Thus, Type 2 diabetes, at least early in the natural progression of the disease is characterized primarily by insulin resistance rather than by a decrease in insulin production, resulting in insufficient uptake, oxidation and storage of glucose in muscle, inadequate repression of lipolysis in adipose tissue, and excess glucose production and secretion by the liver. The net effect of decreased sensitivity to insulin is high levels of insulin circulating in the blood without appropriate reduction in plasma glucose (hyperglycemia). Hyperinsulinemia is a risk factor for developing hypertension and may also contribute to vascular disease.

Glucagon serves as the major regulatory hormone attenuating the effect of insulin in its inhibition of liver gluconeogenesis and is normally secreted by α-cells in pancreatic islets in response to falling blood glucose levels. The hormone binds to specific receptors in liver cells that triggers glycogenolysis and an increase in gluconeogenesis through cAMP-mediated events. These responses generate glucose (e.g. hepatic glucose production) to help maintain euglycemia by preventing blood glucose levels from falling significantly.

In addition to elevated levels of circulating insulin, type II diabetics have elevated levels of plasma glucagon and increased rates of hepatic glucose production. Antagonists of glucagon are useful in improving insulin responsiveness in the liver, decreasing the rate of gluconeogenesis and lowering the rate of hepatic glucose output resulting in a decrease in the levels of plasma glucose.

### SUMMARY OF THE INVENTION

A method of treating type 2 diabetes mellitus in a mammalian patient in need of such treatment comprising administering to said patient an anti-diabetic effective amount of a compound represented by formula I: 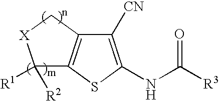
or a pharmaceutically acceptable salt or solvate thereof wherein:

X is CR⁵R⁶;

at least one of R¹, R², R⁵ and R⁶ is present that is other than H;

R¹ is selected from the group consisting of: H, C₁₋₁₀alkyl, C₃₋₇cycloalkyl and Aryl, said alkyl, cycloalkyl and Aryl being optionally substituted with 1-4 substituents independently selected from R¹³;

R² is selected from the group consisting of: R¹ as defined above, —C(O)₂R⁷ and —CONR⁷R⁸;

m and n are selected from 0, 1, 2 and 3, such that the sum of m and n is 2 or 3, and when m is greater than 1, no more than one R¹ and no more than one R² can be other than H;

R³ is selected from the group consisting of: C₁₋₁₀alkyl, C₃₋₇cycloalkyl and Aryl, said alkyl, cycloalkyl and Aryl being optionally substituted with 1-4 substituents selected from R¹³, such that when R³ represents C₁₋₁₀ alkyl substituted with one R¹³ group, and R¹³ represents halo, R¹, R², R⁵ and R⁶ do not represent C₁₋₃alkyl;

R⁵ is selected from the group consisting of: H, C₁₋₁₀alkyl, C₃₋₇cycloalkyl and Aryl, said alkyl, cycloalkyl and Aryl being optionally substituted with 1-4 substituents selected from R¹³;

R⁶ is selected from the group consisting of: R¹ as defined above, HAR, Hetcy, and OR¹¹, wherein said HAR and Hetcy being optionally substituted with 1-4 substituents selected from R¹³,

or R⁵ and R⁶ can be taken in combination with the carbon atom to which they are attached and represent —O—(CH₂)₁₋₂—O— or —C(O)—;

R⁷, R¹⁰ and R¹¹ are selected from the group consisting of: R¹ as defined above, HAR and Hetcy, said HAR and Hetcy being optionally substituted with 1-4 substituents selected from R¹³;

R⁸, R⁹ and R¹² are selected from the group consisting of: C₁₋₁₀alkyl, C₃₋₇cycloalkyl, Aryl, HAR and Hetcy, said alkyl, cycloalkyl, Aryl, HAR and Hetcy being optionally substituted with 1-4 substituents selected from R¹³;

or alternatively, R⁷, R⁸, R⁹ and R¹⁰ are as defined above, and R¹¹ and R¹² are taken together with the atoms to which they are attached and form a 5-8 membered ring optionally containing 1-2 heteroatoms selected from O, S and N, and optionally substituted with 1-4 substituents selected from R¹³;

each R¹³ is selected from the group consisting of: halo, NR¹⁴R¹⁵, C₁₋₄alkyl, C₃₋₇-cycloalkyl, Aryl, HAR, Hetcy, CF₃, OCF₃, OR¹⁵, NO₂, S(O)ₓR¹⁴, SR¹⁴, S(O)_{d}NR¹⁴R¹⁵, O(CR¹⁶R¹⁷)_{y}NR¹⁴R¹⁵, C(O)R¹⁴, CO₂R¹⁵, CO₂(CR¹⁶R¹⁷)_{y}CONR¹⁴R¹⁵, OC(O)R¹⁴, CN, C(O)NR¹⁴R¹⁵, NR¹⁵C(O)R¹⁴, NR¹⁵C(O)OR¹⁴, NR¹⁵C(O)NR¹⁶R¹⁴ and CR¹⁵(N—OR¹⁴),

wherein x is 1 or 2, and y is an integer from 1-4,

said alkyl, cycloalkyl, Aryl, HAR and Hetcy being optionally substituted with 1-4 substituents selected from R¹⁸;

R¹⁴, R¹⁵, R¹⁶ and R¹⁷ are independently selected from the group consisting of: H, C₁₋₁₀alkyl, C₃₋₇cycloalkyl, Aryl and Ar—C₁₋₁₀alkyl;

and each R¹⁸ is independently selected from the group consisting of: halogen, CN, C₁₋₄alkyl, OH, CF₃, Aryl, Aryloxy, CO₂H and CO₂C₁₋₄ alky, said Aryl and the Aryl portion of Aryloxy being optionally substituted with up to 4 halo groups, and up to 2 C₁₋₄ alkyl, OH, CF₃ or CN groups.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, means carbon chains which may be linear, branched, or cyclic, or combinations thereof, containing the indicated number of carbon atoms. If no number is specified, 1-10 carbon atoms are intended for linear or branched alkyl groups. Examples of alkyl groups include methyl, ethyl, propyl, isopropyl, butyl, sec- and tert-butyl, pentyl, hexyl, heptyl, octyl, nonyl and the like. Cycloalkyl is a subset of alkyl; if no number of atoms is specified, 3-10 carbon atoms are intended, forming 1-3 carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Aryl” (Ar) means mono- and bicyclic aromatic rings containing only carbon atoms. Examples of aryl include phenyl and naphthyl.

“Heteroaryl” (HAR) means a mono- or bicyclic aromatic ring or ring system containing at least one heteroatom selected from O, S and N, with each ring containing 5 to 6 atoms. Examples include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl and the like. Heteroaryl also includes aromatic heterocyclic groups fused to heterocycles that are non-aromatic or partially aromatic, and aromatic heterocyclic groups fused to cycloalkyl rings.

“Heterocyclyl” (Hetcy) means mono- and bicyclic saturated rings and ring systems containing at least one heteroatom selected from N, S and O, each of said ring having from 3 to 10 atoms in which the point of attachment may be carbon or nitrogen. Examples of “heterocyclyl” include pyrrolidinyl, piperidinyl, piperazinyl, imidazolidinyl, 2,3-dihydrofuro(2,3-b)pyridyl, benzoxazinyl, tetrahydrohydroquinolinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

“Halogen” (Halo) includes fluorine, chlorine, bromine and iodine. or a pharmaceutically acceptable salt or solvate thereof wherein:

In one aspect of the invention, a method of treating type 2 diabetes mellitus in a mammalian patient in need of such treatment is disclosed which comprises administering to said patient an anti-diabetic effective amount of a compound represented by formula I: 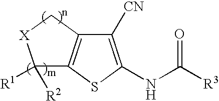
or a pharmaceutically acceptable salt or solvate thereof wherein:

X is CR⁵R⁶;

at least one of R¹, R², R⁵ and R⁶ is present that is other than H;

R¹ is selected from the group consisting of: H, C₁₋₁₀alkyl, C₃₋₇cycloalkyl and Aryl, said alkyl, cycloalkyl and Aryl being optionally substituted with 1-4 substituents independently selected from R¹³;

R² is selected from the group consisting of: R¹ as defined above, —C(O)₂R⁷ and —CONR⁷R⁸;

m and n are selected from 0, 1, 2 and 3, such that the sum of m and n is 2 or 3, and when m is greater than 1, no more than one R¹ and no more than one R² can be other than H;

R³ is selected from the group consisting of: C₁₋₁₀alkyl, C₃₋₇cycloalkyl and Aryl, said alkyl, cycloalkyl and Aryl being optionally substituted with 1-4 substituents selected from R¹³, such that when R³ represents C₁₋₁₀alkyl substituted with one R¹³ group, and R¹³ represents halo, R¹, R², R⁵ and R⁶ do not represent C₁₋₃alkyl;

R⁵ is selected from the group consisting of: H, C₁₋₁₀alkyl, C₃₋₇cycloalkyl and Aryl, said alkyl, cycloalkyl and Aryl being optionally substituted with 1-4 substituents selected from R¹³;

R⁶ is selected from the group consisting of: R¹ as defined above, HAR, Hetcy, and OR¹¹, wherein said HAR and Hetcy being optionally substituted with 1-4 substituents selected from R¹³,

or R⁵ and R⁶ can be taken in combination with the carbon atom to which they are attached and represent —O—(CH₂)₁₋₂—O— or —C(O)—;

R⁷, R¹⁰ and R¹¹ are selected from the group consisting of: R¹ as defined above, HAR and Hetcy, said HAR and Hetcy being optionally substituted with 1-4 substituents selected from R¹³;

R⁸, R⁹ and R¹² are selected from the group consisting of: C₁₋₁₀alkyl, C₃₋₇cycloalkyl, Aryl, HAR and Hetcy, said alkyl, cycloalkyl, Aryl, HAR and Hetcy being optionally substituted with 1-4 substituents selected from R¹³;

or alternatively, R⁷, R⁸, R⁹ and R¹⁰ are as defined above, and R¹¹ and R¹² are taken together with the atoms to which they are attached and form a 5-8 membered ring optionally containing 1-2 heteroatoms selected from O, S and N, and optionally substituted with 1-4 substituents selected from R¹³;

each R¹³ is selected from the group consisting of: halo, NR¹⁴R¹⁵, C₁₋₄alkyl, C₃₋₇-cycloalkyl, Aryl, HAR, Hetcy, CF₃, OCF₃, OR¹⁵, NO₂, S(O)ₓR¹⁴, SR¹⁴, S(O)ₓNR¹⁴R¹⁵, O(CR¹⁶R¹⁷)_{y}NR¹⁴R¹⁵, C(O)R¹⁴, CO₂R¹⁵, CO₂(CR¹⁶R¹⁷)_{y}CONR¹⁴R¹⁵, OC(O)R¹⁴, CN, C(O)NR¹⁴R¹⁵, NR¹⁵C(O)R¹⁴, NR¹⁵C(O)OR¹⁴, NR¹⁵C(O)NR¹⁶R¹⁴ and CR¹⁵(N—OR¹⁴),

wherein x is 1 or 2, and y is an integer from 1-4,

said alkyl, cycloalkyl, Aryl, HAR and Hetcy being optionally substituted with 1-4 substituents selected from R¹⁸;

R¹⁴, R¹⁵, R¹⁶ and R¹⁷ are independently selected from the group consisting of: H, C₁₋₁₀alkyl, C₃₋₇cycloalkyl, Aryl and Ar—C₁₋₁₀alkyl;

and each R¹⁸ is independently selected from the group consisting of: halogen, CN, C₁₋₄alkyl, OH, CF₃, Aryl, Aryloxy, CO₂H and CO₂C₁₋₄ alkyl, said Aryl and the Aryl portion of Aryloxy being optionally substituted with up to 4 halo groups, and up to 2 C₁₋₄ alkyl, OH, CF₃ or CN groups.

In one aspect of the invention that is of particular interest, a method of treating type 2 diabetes mellitus is disclosed wherein a compound of formula I is administered, and R¹ is selected from the group consisting of: H, C₁₋₆alkyl and C₃₋₆cycloalkyl, said alkyl and cycloalkyl being optionally substituted with 1-3 substituents independently selected from R¹³. Within this aspect of the invention, all other variables are as originally defined.

In another aspect of the invention that is of particular interest, a method of treating type 2 diabetes mellitus is disclosed wherein a compound of formula I is administered, and R² is selected from the group consisting of: H, C₁₋₆alkyl, C₃₋₆cycloalkyl, Aryl and C(O)NR⁷R⁸, said alkyl, cycloalkyl and Aryl groups being optionally substituted with 1-3 substituents independently selected from R¹³; R⁷ is selected from the group consisting of: H and C₁₋₆ alkyl, optionally substituted with 1-3 R¹³ groups; R⁸ is selected from the group consisting of: C₁₋₆ alkyl, C₃₋₆ cycloalkyl, and Aryl, optionally substituted with 1-3 R¹³ groups; each R¹³ is independently selected from the group consisting of: halo, Aryl, CF₃ and OCF₃, and Aryl is optionally substituted with 1-3 R¹⁸ groups, which are each independently selected from halo, CH₃, OH, CF₃ and CO₂H. Within this aspect of the invention, all other variables are as originally defined.

In another aspect of the invention that is of particular interest, a method of treating type 2 diabetes mellitus is disclosed wherein a compound of formula I is administered, and R³ is selected from the group consisting of: C₁₋₁₀alkyl and C₃₋₇cycloalkyl, said alkyl and cycloalkyl being optionally substituted with 1-3 substituents selected from R¹³, such that when R³ represents C₁₋₁₀ alkyl substituted with one R¹³ group, and R¹³ represents halo, R¹, R², R⁵and R⁶do not represent C₁₋₃alkyl. Within this aspect of the invention, all other variables are as originally defined.

In another aspect of the invention that is of particular interest, a method of treating type 2 diabetes mellitus is disclosed wherein a compound of formula I is administered, and R⁵ is selected from the group consisting of: H, C₁₋₆alkyl and C₃₋₆cycloalkyl, said alkyl and cycloalkyl being optionally substituted with 1-3 substituents selected from R¹³. Within this aspect of the invention, all other variables are as originally defined.

In another aspect of the invention that is of particular interest, a method of treating type 2 diabetes mellitus is disclosed wherein a compound of formula I is administered, and R⁶ is selected from the group consisting of: H, C₁₋₆alkyl and C₃₋₆cycloalkyl, said alkyl and cycloalkyl being optionally substituted with 1-3 substituents selected from R¹³. Within this aspect of the invention, all other variables are as originally defined.

In another aspect of the invention that is of particular interest, a method of treating type 2 diabetes mellitus is disclosed wherein a compound of formula I is administered and each R¹³ is selected from the group consisting of: halo, C₁₋₄alkyl, C₃₋₆cycloalkyl, Aryl, CF₃ and OCF₃, and Aryl is optionally substituted with 1-3 R¹⁸ groups, which are independently selected from halo, CH₃, OH, CF₃ and CO₂H. Within this aspect of the invention, all other variables are as originally defined.

In an aspect of the invention that is of even more interest, a method of treating type 2 diabetes mellitus is disclosed wherein a compound of formula I is administered, such that:

R¹ is selected from the group consisting of: H, C₁₋₆alkyl and C₃₋₆cycloalkyl, said alkyl and cycloalkyl being optionally substituted with 1-3 substituents independently selected from R¹³;

R² is selected from the group consisting of: H, C₁₋₆alkyl, C₃₋₆cycloalkyl, Aryl and C(O)NR⁷R⁸, said alkyl, cycloalkyl and Aryl groups being optionally substituted with 1-3 substituents independently selected from R¹³;

R⁷ is selected from the group consisting of: H and C₁₋₆ alkyl, optionally substituted with 1-3 R¹³ groups;

R⁸ is selected from the group consisting of: C₁₋₆ alkyl, C₃₋₆ cycloalkyl, and Aryl, optionally substituted with 1-3 R¹³ groups;

each R¹³ is independently selected from the group consisting of: halo, Aryl, CF₃ and OCF₃, and Aryl is optionally substituted with 1-3 R¹⁸ groups, which are each independently selected from halo, CH₃, OH, CF₃ and CO₂H;

R³ is selected from the group consisting of: C₁₋₁₀alkyl and C₃₋₇cycloalkyl, said alkyl and cycloalkyl being optionally substituted with 1-3 substituents selected from R¹³, such that when R³ represents C₁₋₁₀ alkyl substituted with one R¹³ group, and R¹³ represents halo, R¹, R², R⁵ and R⁶ do not represent C₁₋₃alkyl;

R⁵ is selected from the group consisting of: H, C₁₋₆alkyl and C₃₋₆cycloalkyl, said alkyl and cycloalkyl being optionally substituted with 1-3 substituents selected from R¹³;

R⁶ is selected from the group consisting of: H, C₁₋₆alkyl and C₃₋₆cycloalkyl, said alkyl and cycloalkyl being optionally substituted with 1-3 substituents selected from R¹³, and

each R¹³ is selected from the group consisting of: halo, C₁₋₄alkyl, C₃₋₆cycloalkyl, Aryl, CF₃ and OCF₃, and Aryl is optionally substituted with 1-3 R¹⁸ groups, which are independently selected from halo, CH₃, OH, CF₃ and CO₂H. Within this aspect of the invention, all other variables are as originally defined.

Also included herein is a pharmaceutical composition that is comprised of a compound represented by formula I: 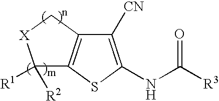
or a pharmaceutically acceptable salt or solvate thereof in combination with a pharmaceutically acceptable carrier, wherein:

X is CR⁵R⁶;

at least one of R¹, R², R⁵ and R⁶ is present that is other than H;

R¹ is selected from the group consisting of: H, C₁₋₁₀alkyl, C₃₋₇cycloalkyl and Aryl, said alkyl, cycloalkyl and Aryl being optionally substituted with 1-4 substituents independently selected from R¹³;

R² is selected from the group consisting of: R¹ as defined above, —C(O)₂R⁷ and —CONR⁷R⁸;

m and n are selected from 0, 1, 2 and 3, such that the sum of m and n is 2 or 3, and when m is greater than 1, no more than one R¹ and no more than one R² can be other than H;

R³ is selected from the group consisting of: C₁₋₁₀alkyl, C₃₋₇cycloalkyl and Aryl, said alkyl, cycloalkyl and Aryl being optionally substituted with 1-4 substituents selected from R¹³, such that when R³ represents C₁₋₁₀alkyl substituted with one R¹³ group, and R¹³ represents halo, R¹, R², R⁵ and R⁶ do not represent C₁₋₃alkyl;

R⁵ is selected from the group consisting of: H, C₁₋₁₀alkyl, C₃₋₇cycloalkyl and Aryl, said alkyl, cycloalkyl and Aryl being optionally substituted with 1-4 substituents selected from R¹³;

R⁶ is selected from the group consisting of: R¹ as defined above, HAR, Hetcy, and OR¹¹, wherein said HAR and Hetcy being optionally substituted with 1-4 substituents selected from R¹³,

or R⁵ and R⁶ can be taken in combination with the carbon atom to which they are attached and represent —O—(CH₂)₁₋₂—O— or —C(O)—;

R⁷, R¹⁰ and R¹¹ are selected from the group consisting of: R¹ as defined above, HAR and Hetcy, said HAR and Hetcy being optionally substituted with 1-4 substituents selected from R¹³;

R⁸, R⁹ and R¹² are selected from the group consisting of: C₁₋₁₀alkyl, C₃₋₇cycloalkyl, Aryl, HAR and Hetcy, said alkyl, cycloalkyl, Aryl, HAR and Hetcy being optionally substituted with 1-4 substituents selected from R¹³;

or alternatively, R⁷, R⁸, R⁹ and R¹⁰ are as defined above, and R¹¹ and R¹² are taken together with the atoms to which they are attached and form a 5-8 membered ring optionally containing 1-2 heteroatoms selected from O, S and N, and optionally substituted with 1-4 substituents selected from R¹³;

each R¹³ is selected from the group consisting of: halo, NR¹⁴R¹⁵, C₁₋₄alkyl, C₃₋₇-cycloalkyl, Aryl, HAR, Hetcy, CF₃, OCF₃, OR¹⁵, NO₂, S(O)ₓR¹⁴, SR¹⁴, S(O)ₓNR¹⁴R¹⁵, O(CR¹⁶R¹⁷)_{y}NR¹⁴R¹⁵, C(O)R¹⁴, CO₂R¹⁵, CO₂(CR¹⁶R¹⁷)_{y}CONR¹⁴R¹⁵, OC(O)R¹⁴, CN, C(O)NR¹⁴R¹⁵, NR¹⁵C(O)R¹⁴, NR¹⁵C(O)OR¹⁴, NR¹⁵C(O)NR¹⁶R¹⁴ and CR¹⁵(N—OR¹⁴),

wherein x is 1 or 2, and y is an integer from 1-4,

said alkyl, cycloalkyl, Aryl, HAR and Hetcy being optionally substituted with 1-4 substituents selected from R¹⁸;

R¹⁴, R¹⁵, R¹⁶ and R¹⁷ are independently selected from the group consisting of: H, C₁₋₁₀alkyl, C₃₋₇cycloalkyl, Aryl and Ar—C₁₋₁₀alkyl;

and each R¹⁸ is independently selected from the group consisting of: halogen, CN, C₁₋₄alkyl, OH, CF₃, Aryl, Aryloxy, CO₂H and CO₂C₁₋₄ alkyl, said Aryl and the Aryl portion of Aryloxy being optionally substituted with up to 4 halo groups, and up to 2 C₁₋₄ alkyl, OH, CF₃ or CN groups.

More particularly, a pharmaceutical composition is disclosed which is comprising of a compound of formula I wherein R¹ is selected from the group consisting of: H, C₁₋₆alkyl and C₃₋₆cycloalkyl, said alkyl and cycloalkyl being optionally substituted with 1-3 substituents independently selected from R¹³ in combination with a pharmaceutically acceptable carrier. Within this aspect of the invention, all other variables are as originally defined.

In another aspect of the invention that is of particular interest, the pharmaceutical composition is comprised of a compound of formula I wherein R² is selected from the group consisting of: H, C₁₋₆alkyl, C₃₋₆cycloalkyl, Aryl and C(O)NR⁷R⁸, said alkyl, cycloalkyl and Aryl groups being optionally substituted with 1-3 substituents independently selected from R¹³; R⁷ is selected from the group consisting of: H and C₁₋₆ alkyl, optionally substituted with 1-3 R¹³ groups; R⁸ is selected from the group consisting of: C₁₋₆ alkyl, C₃₋₆ cycloalkyl, and Aryl, optionally substituted with 1-3 R¹³ groups; each R¹³ is independently selected from the group consisting of: halo, Aryl, CF₃ and OCF₃, and Aryl is optionally substituted with 1-3 R¹⁸ groups, which are each independently selected from halo, CH₃, OH, CF₃ and CO₂H. Within this aspect of the invention, all other variables are as originally defined.

In another aspect of the invention that is of particular interest, the pharmaceutical composition is comprised of a compound of formula I wherein R³ is selected from the group consisting of: C₁₋₁₀alkyl and C₃₋₇cycloalkyl, said alkyl and cycloalkyl being optionally substituted with 1-3 substituents selected from R¹³, such that when R³ represents C₁₋₁₀ alkyl substituted with one R¹³ group, and R¹³ represents halo, R¹, R², R⁵ and R⁶ do not represent C₁₋₃alkyl. Within this aspect of the invention, all other variables are as originally defined.

In another aspect of the invention that is of particular interest, a pharmaceutical composition is comprised of a compound of formula I wherein R⁵ is selected from the group consisting of: H, C₁₋₆alkyl and C₃₋₆cycloalkyl, said alkyl and cycloalkyl being optionally substituted with 1-3 substituents selected from R¹³. Within this aspect of the invention, all other variables are as originally defined.

In another aspect of the invention that is of particular interest, the pharmaceutical composition is comprised of a compound of formula I wherein R⁶ is selected from the group consisting of: H, C₁₋₆alkyl and C₃₋₆cycloalkyl, said alkyl and cycloalkyl being optionally substituted with 1-3 substituents selected from R¹³. Within this aspect of the invention, all other variables are as originally defined.

In another aspect of the invention that is of particular interest, the pharmaceutical composition is comprised of a compound of formula I wherein each R³ is selected from the group consisting of: halo, C₁₋₄alkyl, C₃₋₆cycloalkyl, Aryl, CF₃ and OCF₃, and Aryl is optionally substituted with 1-3 R¹⁸ groups, which are independently selected from halo, CH₃, OH, CF₃ and CO₂H. Within this aspect of the invention, all other variables are as originally defined.

In an aspect of the invention that is of even more interest, a pharmaceutical composition is disclosed which is comprised of a compound formula I wherein:

R¹ is selected from the group consisting of: H, C₁₋₆alkyl and C₃₋₆cycloalkyl, said alkyl and cycloalkyl being optionally substituted with 1-3 substituents independently selected from R¹³;

R² is selected from the group consisting of: H, C₁₋₆alkyl, C₃₋₆cycloalkyl, Aryl and C(O)NR⁷R⁸, said alkyl, cycloalkyl and Aryl groups being optionally substituted with 1-3 substituents independently selected from R¹³;

R⁷ is selected from the group consisting of: H and C₁₋₆ alkyl, optionally substituted with 1-3 R¹³ groups;

R⁸ is selected from the group consisting of: C₁₋₆ alkyl, C₃₋₆ cycloalkyl, and Aryl, optionally substituted with 1-3 R¹³ groups;

each R¹³ is independently selected from the group consisting of: halo, Aryl, CF₃ and OCF₃, and Aryl is optionally substituted with 1-3 R¹⁸ groups, which are each independently selected from halo, CH₃, OH, CF₃ and CO₂H;

R³ is selected from the group consisting of: C₁₋₁₀alkyl and C₃₋₇cycloalkyl, said alkyl and cycloalkyl being optionally substituted with 1-3 substituents selected from R¹³, such that when R³ represents C₁₋₁₀ alkyl substituted with one R¹³ group, and R¹³ represents halo, R¹, R², R⁵ and R⁶ do not represent C₁₋₃alkyl;

R⁵ is selected from the group consisting of: H, C₁₋₆alkyl and C₃₋₆cycloalkyl, said alkyl and cycloalkyl being optionally substituted with 1-3 substituents selected from R¹³;

R⁶ is selected from the group consisting of: H, C₁₋₆alkyl and C₃₋₆cycloalkyl, said alkyl and cycloalkyl being optionally substituted with 1-3 substituents selected from R¹³, and

each R¹³ is selected from the group consisting of: halo, C₁₋₄alkyl, C₃₋₆cycloalkyl, Aryl, CF₃ and OCF₃, and Aryl is optionally substituted with 1-3 R¹⁸ groups, which are independently selected from halo, CH₃, OH, CF₃ and CO₂H. Within this aspect of the invention, all other variables are as originally defined.

Also included is a method of preventing or delaying the onset of type 2 diabetes mellitus in a mammalian patient in need of such treatment, comprising administering to said patient a compound represented by formula I: 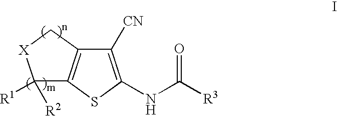
or a pharmaceutically acceptable salt or solvate thereof wherein:

X is CR⁵R⁶;

at least one of R¹, R², R⁵ and R⁶ is present that is other than H;

R¹ is selected from the group consisting of: H, C₁₋₁₀alkyl, C₃₋₇cycloalkyl and Aryl, said alkyl, cycloalkyl and Aryl being optionally substituted with 1-4 substituents independently selected from R¹³;

R² is selected from the group consisting of: R¹ as defined above, —C(O)₂R⁷ and —CONR⁷R⁸;

m and n are selected from 0, 1, 2 and 3, such that the sum of m and n is 2 or 3, and when m is greater than 1, no more than one R¹ and no more than one R² can be other than H;

R³ is selected from the group consisting of: C₁₋₁₀alkyl, C₃₋₇cycloalkyl and Aryl, said alkyl, cycloalkyl and Aryl being optionally substituted with 1-4 substituents selected from R¹³, such that when R³ represents C₁₋₁₀alkyl substituted with one R¹³ group, and R¹³ represents halo, R¹, R², R⁵ and R⁶ do not represent C₁₋₃alkyl;

R⁵ is selected from the group consisting of: H, C₁₋₁₀alkyl, C₃₋₇cycloalkyl and Aryl, said alkyl, cycloalkyl and Aryl being optionally substituted with 1-4 substituents selected from R¹³;

R⁶ is selected from the group consisting of: R¹ as defined above, HAR, Hetcy, and OR¹¹, wherein said HAR and Hetcy being optionally substituted with 1-4 substituents selected from R¹³,

or R⁵ and R⁶ can be taken in combination with the carbon atom to which they are attached and represent —O—(CH₂)₁₋₂—O— or —C(O)—;

R⁷, R¹⁰ and R¹¹ are selected from the group consisting of: R¹ as defined above, HAR and Hetcy, said HAR and Hetcy being optionally substituted with 1-4 substituents selected from R¹³;

R⁸, R⁹ and R¹² are selected from the group consisting of: C₁₋₁₀alkyl, C₃₋₇cycloalkyl, Aryl, HAR and Hetcy, said alkyl, cycloalkyl, Aryl, HAR and Hetcy being optionally substituted with 1-4 substituents selected from R¹³;

or alternatively, R⁷, R⁸, R⁹ and R¹⁰ are as defined above, and R¹¹ and R¹² are taken together with the atoms to which they are attached and form a 5-8 membered ring optionally containing 1-2 heteroatoms selected from O, S and N, and optionally substituted with 1-4 substituents selected from R¹³;

each R¹³ is selected from the group consisting of: halo, NR¹⁴R¹⁵, C₁₋₄alkyl, C₃₋₇₋cycloalkyl, Aryl, HAR, Hetcy, CF₃, OCF₃, OR¹⁵, NO₂, S(O)ₓR¹⁴, SR¹⁴, S(O)ₓNR¹⁴R¹⁵, O(CR¹⁶R¹⁷)_{y}NR¹⁴R¹⁵, C(O)R¹⁴, CO₂R¹⁵, CO₂(CR¹⁶R¹⁷)_{y}CONR¹⁴R¹⁵, OC(O)R¹⁴, CN, C(O)NR¹⁴R¹⁵, NR¹⁵C(O)R¹⁴, NR¹⁵C(O)OR¹⁴, NR¹⁵C(O)NR¹⁶R¹⁴ and CR¹⁵(N—OR¹⁴),

wherein x is 1 or 2, and y is an integer from 1-4,

said alkyl, cycloalkyl, Aryl, HAR and Hetcy being optionally substituted with 1-4 substituents selected from R¹⁸;

R¹⁴, R¹⁵, R¹⁶ and R¹⁷ are independently selected from the group consisting of: H, C₁₋₁₀alkyl, C₃₋₇cycloalkyl, Aryl and Ar-C₁₋₁₀alkyl;

and each R¹⁸ is independently selected from the group consisting of: halogen, CN, C₁₋₄alkyl, OH, CF₃, Aryl, Aryloxy, CO₂H and CO₂C₁₋₄ alkyl, said Aryl and the Aryl portion of Aryloxy being optionally substituted with up to 4 halo groups, and up to 2 C₁₋₄ alkyl, OH, CF₃ or CN groups,

said compound being administered in an amount that is effective to prevent or delay the onset of type 2 diabetes mellitus.

Also included is a method of treating, preventing or delaying the onset of a disease or condition in a type 2 diabetes mellitus patient, said disease or condition being selected from the group consisting of: dyslipidemia selected from elevated serum cholesterol, elevated serum triglycerides, elevated serum low density lipoproteins and low levels of serum high density lipoprotein, microvascular or macrovascular changes and the sequellae of such conditions selected from coronary heart disease, stroke, peripheral vascular disease, hypertension, renal hypertension, nephropathy, neuropathy and retinopathy, said method comprising administering to the type 2 diabetic patient an amount of a compound of formula I that is effective for treating, preventing or delaying the onset of such disease or condition.

Examples of species that are of particular interest in the methods and compositions described herein include the following: N-(3-cyano-6-methyl-4,5,6,7-tetrahydro-1-benzothien-2-yl)-2-ethylbutanamide; N-(3-cyano-6-tert-pentyl-4,5,6,7-tetrahydro-1-benzothien-2-yl)-2-ethylbutanamide; N-(3-cyano-6-tert-pentyl-4,5,6,7-tetrahydro-1-benzothien-2-yl)-3-methylbutanamide; N-(6-tert-butyl-3-cyano-4,5,6,7-tetrahydro-1-benzothien-2-yl)decanamide; N-(3-cyano-6-tert-pentyl-4,5,6,7-tetrahydro-1-benzothien-2-yl)bicyclo[2.2.1]heptane-2-carboxamide; N-(3-cyano-6-ethyl-4,5,6,7-tetrahydro-1-benzothien-2-yl)-2-phenylcyclopropanecarboxamide; N-(3-cyano-6-tert-pentyl-4,5,6,7-tetrahydro-1-benzothien-2-yl)-2-phenylcyclopropanecarboxamide; N-(3-cyano-6-tert-pentyl-4,5,6,7-tetrahydro-1-benzothien-2-yl)cyclopentanecarboxamide; N-(3-cyano-6-tert-pentyl-4,5,6,7-tetrahydro-1-benzothien-2-yl)-2,2,3,3-tetramethylcyclopropanecarboxamide; N-(3-cyano-6-tert-pentyl-4,5,6,7-tetrahydro-1-benzothien-2-yl)-3-cyclohexylpropanamide; N-(3-cyano-6-tert-pentyl-4,5,6,7-tetrahydro-1-benzothien-2-yl)-3-phenylpropanamide; N-(3-cyano-6-tert-pentyl-4,5,6,7-tetrahydro-1-benzothien-2-yl)-3,3-dimethylbutanamide; N-(3-cyano-6-tert-pentyl-4,5,6,7-tetrahydro-1-benzothien-2-yl)-4,4,4-trifluoro-3-methylbutanamide; N-(3-cyano-6-tert-pentyl-4,5,6,7-tetrahydro-1-benzothien-2-yl)-2,2-dimethylpropanamide; N-(6-tert-butyl-3-cyano-4,5,6,7-tetrahydro-1-benzothien-2-yl)cyclopentanecarboxamide; N-(3-cyano-6-phenyl-4,5,6,7-tetrahydro-1-benzothien-2-yl)cyclopentanecarboxamide; N-(3-cyano-6-phenyl-4,5,6,7-tetrahydro-1-benzothien-2-yl)-2-ethylbutanamide; N-(3-cyano-5,5,7,7-tetramethyl-4,5,6,7-tetrahydro-1-benzothien-2-yl)-2-ethylbutanamide; N-(3-cyano-5-tert-pentyl-5,6-dihydro-4H-cyclopenta[b]thien-2-yl)-2-ethylbutanamide; N-(3-cyano-6-tert-pentyl-5,6-dihydro-4H-cyclopenta[b]thien-2-yl)-2-ethylbutanamide; N-(3-cyano-4,6-dimethyl-5,6-dihydro4H-cyclopenta[b]thien-2-yl)-2-ethylbutanamide; N-(3-cyano-7-phenyl-4,5,6,7-tetrahydro-1-benzothien-2-yl)-2-ethylbutanamide; and 3-cyano-N-(2,4-dichlorobenzyl)-2-[(2-ethylbutanoyl)amino]-N-isopropyl-4,5,6,7-tetrahydro-1-benzothiophene-7-carboxamide

Also included in a method of treating, preventing or delaying the onset of diseases or conditions that are associated with type 2 diabetes mellitus. Examples include diseases and conditions selected from the group consisting of: dyslipidemias, such as elevated levels of cholesterol, triglycerides or low density lipoproteins (LDL), low levels of high density lipoprotein (HDL), microvascular or macrovascular changes and the sequellae of such conditions, such as coronary heart disease, stroke, peripheral vascular disease, hypertension, renal hypertension, nephropathy, neuropathy and retinopathy. The method entails administering to a type 2 diabetic patient, e.g., a human patient, an amount of a compound of formula I that is effective for treating, preventing or delaying the onset of such diseases or conditions.

Optical Isomers-Diastereomers-Geometric Isomers-Tautomers

Many of the compounds of formula I contain one or more asymmetric centers and thus occur as racemates and racemic mixtures, single enantiomers, diastereomeric mixtures and individual diastereomers. The present invention includes all such isomeric forms of the compounds, in pure form as well as in mixtures.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I.

Salts and Solvates

The term “pharmaceutically acceptable salts” refers to salts prepared from pharmaceutically acceptable substantially non-toxic bases or acids including inorganic or organic bases and inorganic or organic acids, as well as salts that can be converted into pharmaceutically acceptable salts. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

Solvates as used herein refers to the compound of formula I or a salt thereof, in association with a solvent, such as water. Representative examples include hydrates, hemihydrates, trihydrates and the like.

References to the compounds of Formula I include the pharmaceutically acceptable salts and solvates.

This invention relates to method of antagonizing or inhibiting the production or activity of glucagon, thereby reducing the rate of gluconeogenesis and glycogenolysis, and the concentration of glucose in plasma.

The compounds of formula I can be used in the manufacture of a medicament for the prevention of, or prophylactic or therapeutic treatment of type 2 diabetes mellitus or a disease or condition associated therewith, by combining the compound of formula I with the carrier materials.

Dose Ranges

The prophylactic or therapeutic dose of a compound of Formula I will, of course, vary with the nature of the condition to be treated, the particular compound selected and its route of administration. It will also vary according to the age, weight and response of the individual patient. In general, the daily dose range lie within the range of from about 0.001 mg to about 100 mg per kg body weight, preferably about 0.01 mg to about 50 mg per kg, and more preferably 0.1 to 10 mg per kg, in single or divided doses. It may be necessary to use dosages outside of these limits in some cases.

When intravenous or or oral administration is employed, a representative dosage range is from about 0.001 mg to about 100 mg (preferably from 0.01 mg to about 10 mg) of a compound of Formula I per kg of body weight per day, and more preferably, about 0.1 mg to about 10 mg of a compound of Formula I per kg of body weight per day.

Pharmaceutical Compositions

As mentioned above, the pharmaceutical composition comprises a compound of Formula I and a pharmaceutically acceptable carrier. The term “composition” encompasses a product comprising the active and inert ingredient(s), (pharmaceutically acceptable excipients) that make up the carrier, as well as any product which results, directly or indirectly, from the combination, complexation or aggregation of any two or more of the ingredients, or from dissociation of one or more of the ingredients, or from other types of reactions or interactions between ingredients. Preferably the composition is comprised of a compound of formula I in an amount that is effective to treat, prevent or delay the onset of type 2 diabetes mellitus, in combination with the pharmaceutically acceptable carrier.

Any suitable route of administration may be employed for providing a mammal, especially a human with an effective dosage of a compound of the present invention. For example, oral, rectal, topical, parenteral, ocular, pulmonary, nasal, and the like may be employed. Examples of dosage forms include tablets, troches, dispersions, suspensions, solutions, capsules, creams, ointments, aerosols and the like, with oral tablets being preferred.

In preparing oral compositions, any of the usual pharmaceutical media may be employed, such as, for example, water, glycols, oils, alcohols, flavoring agents, preservatives, coloring agents and the like in the case of oral liquids, e.g., suspensions, elixirs and solutions; or carriers such as starches, sugars, microcrystalline cellulose, diluents, granulating agents, lubricants, binders, disintegrating agents and the like in the case of oral solids, e.g., powders, capsules and tablets, with the solid oral preparations being preferred. Because of their ease of administration, tablets and capsules represent the most advantageous oral dosage unit forms. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product appropriately. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 1 g of the active ingredient and each cachet or capsule contains from about 1 to about 500 mg of the active ingredient.

The following are examples of pharmaceutical dosage forms for the compounds of Formula I:

| Injectable Suspension (I.M.) | mg/mL | |
|---|---|---|
| Compound of Formula I | 10 | |
| Methylcellulose | 5.0 | |
| Tween 80 | 0.5 | |
| Benzyl alcohol | 9.0 | |
| Benzalkonium chloride | 1.0 | |
| Water for injection to make | 1.0 | mL |
| Capsule | mg/capsule | |
| Compound of Formula I | 25 | |
| Lactose Powder | 573.5 | |
| Magnesium Stearate | 1.5 | |
| Total | 600 | mg |
| Tablet | mg/tablet | |
| Compound of Formula I | 25 | |
| Microcrystalline Cellulose | 415 | |
| Povidone | 14.0 | |
| Pregelatinized Starch | 43.5 | |
| Magnesium Stearate | 2.5 | |
| Total | 500 | mg |
| Aerosol | Per canister | |
| Compound of Formula I | 24 | mg |
| Lecithin, NF Liq. Conc. | 1.2 | mg |
| Trichlorofluoromethane, NF | 4.025 | g |
| Dichlorodifluoromethane, NF | 12.15 | g |

Combination Therapy

Compounds of Formula I may be used in combination with other drugs that are used in the treatment/prevention/delaying the onset of type 2 diabetes mellitus, as well as the diseases and conditions associated with type 2 diabetes mellitus, for which compounds of Formula I are useful. Other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (a) bis-guanides (e.g., buformin, metformin, phenformin), (b) PPAR agonists (e.g., troglitazone, pioglitazone, rosiglitazone), (c) insulin, (d) somatostatin, (e) α-glucosidase inhibitors (e.g., voglibose, miglitol, acarbose), and (f) insulin secretagogues (e.g., acetohexamide, carbutamide, chlorpropamide, glibornuride, gliclazide, glimerpiride, glipizide, gliquidine, glisoxepid, glyburide, glyhexamide, glypinamide, phenbutamide, tolazamide, tolbutamide, tolcyclamide, nateglinide, repaglinide).

The weight ratio of the compound of the Formula I to the second active ingredient may be varied within wide limits and depends upon the effective dose of each ingredient. Generally, an effective dose of each will be used. Thus, for example, when a compound of the Formula I is combined with a PPAR agonist the weight ratio of the compound of the Formula I to the PPAR agonist will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

Throughout the instant application, the following abbreviations are used with the following meanings unless otherwise indicated:

| | |
|---|---|
| Bu = butyl | Bn = benzyl |
| BOC, Boc = t-butyloxycarbonyl | CBZ, Cbz = Benzyloxycarbonyl |
| DCC = Dicyclohexylcarbodiimide | DCM = dichloromethane |
| DIEA = diisopropylethylamine | DMF = N,N-dimethylformamide |
| DMAP = 4-Dimethylaminopyridine | Et = ethyl |
| EtOAc = ethyl acetate | EtOH = ethanol |
| eq. = equivalent(s) | FAB-mass spectrum = Fast atom |
| | bombardment-mass spectroscopy |
| HOAc = acetic acid | HPLC = High pressure liquid |
| | chromatography |
| HOBT, HOBt = Hydroxybenztriazole | LAH = Lithium aluminum hydride |
| Me = methyl | PBS = phosphate buffer saline |
| Ph = phenyl | TFA = Trifluoroacetic acid |
| THF = Tetrahydrofuran | TMS = Trimethylsilane |

Compounds of the present invention may be prepared according to the methodology outlined in the following Schemes.

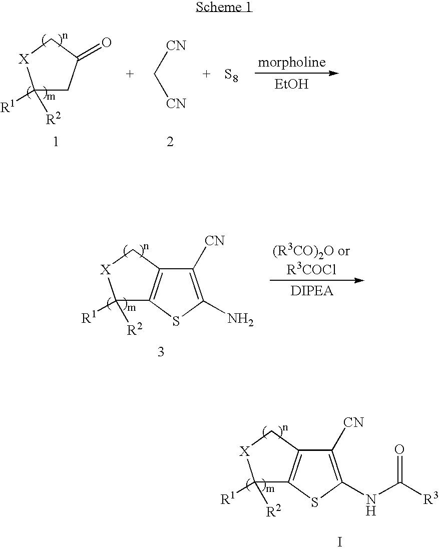

Cyclic ketones such as 1, where X is CR⁵R⁶ from formula I, are commercially available, known in the literature or may be conveniently prepared by a variety of methods familiar to those skilled in the art.

In Scheme 1, a cyclic ketone 1 is condensed with malonitrile 2 in the presence of sulfur (S₈) and a dialkylamine (e.g., morpholine) in ethanol according to methods described in the literature (S. Mukherjee and A. De, J. Chem. Res. 8, 295 (1994); M. S. Mahas et al. J. Chem. Soc. 1969, 1937; A. De et al. J. Het. Chem. 29, 1213 (1992)) to afford 2-amino-3-cyano-thiophene 3. Acylation of 3 with an appropriate anhydride or acid chloride in the presence of a trialkylamine (e.g., diisopropylethylamine) according to published procedures (U. Sensfuss et al. Heteroat. Chem. 9, 529 (1998) will afford the amide represented by formula I.

It is recognized that when the cyclic ketone 1 is not a symmetrically substituted ketone, the product 3 may be formed as a mixture of positional isomers. These isomers may be separated at any stage in the synthetic sequence by preparative thin layer chromatography, flash chromatography on silica gel as described by W. C. Still et al., J. Org. Chem., 43, 2923 (1978), or HPLC. Compounds that are purified by HPLC may be isolated as the corresponding salt.

In some instances it may be necessary to carry out the thiophene synthesis in two steps, as illustrated in Scheme 2.

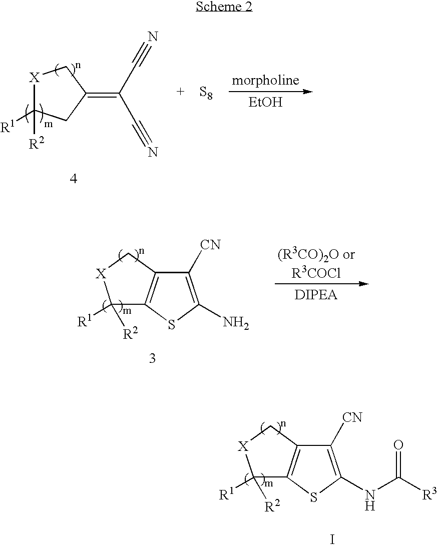

A dicyano-alkene 4 is first prepared by condensation of a ketone such as 1 and malonitrile. This intermediate is reacted with sulfur (S₈) and a dialkylamine (e.g., morpholine) in ethanol according to methods described in the literature (A. Rajca and M. Tisler, Monatch. Chem. 121, 697 (1990); B. Naumann et al., Pharmazie 53, 4 (1996)) to afford 2-amino-3-cyano-thiophene 3. Acylation of 3 with an appropriate anhydride or acid chloride in the presence of a trialkylamine (e.g., diisopropylethylamine) according to published procedures (U. Sensfuss et al. Heteroat. Chem. 9, 529 (1998) afford the thiopheneamide represented by formula I.

The following examples are illustrative of the present invention, and are not to be construed as limiting the scope of the appended claims.

The compounds listed in Table 1 illustrate the present invention and are commercially available from Olivia Scientific, Inc., 475 Wall Street, Princeton, N.J. 08540.

TABLE 1

| Example | |
|---|---|
| No. | Name |
| 1) | N-(3-Cyano-6-methyl-4,5,6,7-tetrahydro-1-benzothien-2-yl)-2- |
| | ethylbutanamide |
| 2) | N-(3-Cyano-6-tert-pentyl-4,5,6,7-tetrahydro-1-benzothien- |
| | 2-yl)-2-ethylbutanamide |
| 3) | N-(3-Cyano-6-tert-pentyl-4,5,6,7-tetrahydro-1-benzothien- |
| | 2-yl)-3-methylbutanamide |
| 4) | N-(6-tert-Butyl-3-cyano-4,5,6,7-tetrahydro-1-benzothien-2- |
| | yl)decanamide |
| 5) | N-(3-Cyano-6-tert-pentyl-4,5,6,7-tetrahydro-1-benzothien-2- |
| | yl)bicyclo[2.2.1]heptane-2-carboxamide |
| 6) | N-(3-Cyano-6-ethyl-4,5,6,7-tetrahydro-1-benzothien-2-yl)-2- |
| | phenylcyclopropanecarboxamide |
| 7) | N-(3-Cyano-6-tert-pentyl-4,5,6,7-tetrahydro-1-benzothien- |
| | 2-yl)-2-phenylcyclopropanecarboxamide |

### EXAMPLE 8

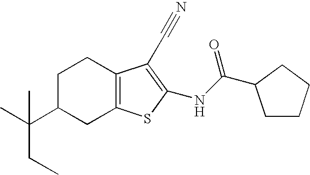

### N-(3-Cyano-6-tert-pentyl-4,5,6,7-tetrahydro-1-benzothien-2-yl)cyclopentanecarboxamide

### Step A. 2-Amino-6-tert-pentyl-4,5,6,7-tetrahydro-1-benzothiophene-3-carbonitrile

The title compound was prepared via the sequence outlined in Scheme 1. To 4-tert-pentylcyclohexanone in 10 mL of EtOH was added 1.97 g (29.8 mmol) of malononitrile, followed by 3.89 mL (44.6 mmol) of morpholine, then 1.90 g (59.5 mmol) of elemental sulfur. The mixture was stirred at ambient temperature for 16 h, then diluted with an equal volume of saturated aqueous NaHCO₃. The mixture was extracted twice with dichloromethane, and the combined organic layers were dried (Na₂SO₄) and concentrated in vacuo. Purification by flash chromatography (20% EtOAc in hexane) afforded the title compound.

¹H NMR (500 MHz, CDCl₃) 4.57 (s, 2H), 2.66 (m, 1H), 2.49 (m, 1H), 2.44 (m, 1H), 2.32 (m, 1H), 1.97 (m, 1H), 1.63 (m, 1H), 1.35 (m, 2H), 0.89 (s, 3H), 0.87 (s, 3H), 8.85 (t, J=7.5 Hz, 3H); mass spectrum (ES) m/e=249 (M+1).

### Step B. N-(3-Cyano-6-tert-pentyl-4,5,6,7-tetrahydro-1-benzothien-2-yl)cyclopentanecarboxamide

To the intermediate prepared in Step A in 4 mL of dichloromethane was added 0.100 mL (0.600 mmol) of di-iso-propylethylamine, followed by 0.080 mL (0.600 mmol) of cyclopenanecarbonyl chloride. After 3 d at ambient temperature, the mixture was diluted with an equal volume of saturated aqueous NaHCO₃ and extracted twice with dichloromethane. The combined organic layers were dried (Na₂SO₄) and concentrated in vacuo. Purification by flash chromatography (6% EtOAc in hexane) afforded the title compound as a white solid.

¹H NMR (500 MHz, CDCl₃) 8.40 (s, 1H), 2.79 (quint., J=8.0 Hz, 1H), 2.71 (dd, J=5.0 Hz, J=16.5 Hz, 1H), 2.60 (dd, J=5.0 Hz, J=16.0 Hz, 1H), 2.46 (m, 1H), 2.37 (m, 1H), 1.98 (m, 3H), 1.87 (m, 2H), 1.78 (m, 2H), 1.62 (m, 3H), 0.86 (s, 3H), 0.85 (t, J=7.5 Hz, 3H); mass spectrum (ES) m/e=345.2 (M+1).

Using the intermediate prepared in Example 8 Step A, and following the procedure outlined in Example 8 Step B, the compounds listed in Table 2 were prepared.

TABLE 2

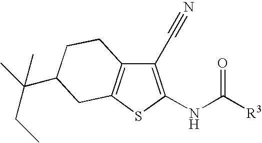

| | | MS (ES) | |
|---|---|---|---|
| Example | R³ | m/e | ¹H NMR (500 MHz) data |
| 9 | 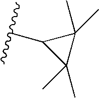 | 373.1 (M + 1) | (CDCl₃) 8.40 (s, 1H), 2.70 (dd, J = 4.5 Hz, J = 16.5 Hz, 1H), 2.57 (dd, J = 4.5 Hz, J = 16.0 Hz, 1H), 2.46 (m, 1H), 2.34 (m, 1H), 1.97 (dd, J = 2.5 Hz, J =13 Hz, 1H), 1.57 (dt, J = 3.0 Hz, J =11.5 Hz, 1H), 1.32 (m, 1H), 1.30 (s, 6H), 1.18 (s, 6H), 1.12 (s, 1H), 0.86 (s, 3H), 0.84 (s, 3H), 0.81 (t, J = 7.5 Hz, 3H) |
| 10 | 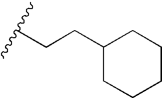 | 387.3 (M + 1) | (CDCl₃) 8.43 (s, 1H), 2.72 (dd, J = 5.0 Hz, J = 16.5 Hz, 1H), 2.61 (dd, J = 4.5 Hz J = 16.0 Hz, 1H), 2.46 (t, J = 7.5 Hz, 2H), 2.37 (m, 1H), 1.98 (dd, J =3.5 Hz, J = 13 Hz, 1H), 1.71 (m, 2H), 1.62 (m, 3H), 1.33 (m, 3H), 1.21 (m, 2H), 0.93 (m, 1H), 0.87 (s, 3H), 0.83 (s, 3H), 0.82 (t, J = 7.5 Hz, 3H) |
| 11 | 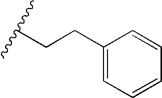 | 381.2 (M + H) | (CDCl₃) 8.59 (s, 1H), 7.27 (m, 2H), 7.22 (m, 3H), 3.05 (t, J = 7.5 Hz, 2H), 2.78 (t, J = 7.5 Hz, 2H), 2.69 (m, 1H), 2.61 (m, 1H), 2.44 (m, 1H), 2.38 (m, 1H), 1.97 (m, 1H), 1.58 (m, 1H), 1.33 (m, 2H), 0.87 (s, 3H), 0.85 (s, 3H), 0.82 (t, J = 7.5 Hz, 3H) |
| 12 | 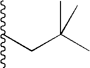 | 347.1 (M + H) | (CDCl₃) 8.99 (s, 1H), 2.70 (dd, J = 4.5 Hz, J = 16.0 Hz, 1H), 2.60 (dd, J = 5.0 Hz, J = 16.0 Hz, 1H) 2.46 (dt, J = 3.5 Hz, J = 8.5 Hz, 1H), 2.38 (m, 1H), 2.34 (s, 2H), 1.98 (dd, J = 3.5 Hz, J =12.5 Hz, 1H), 1.57 (dt, J = 3.5 Hz, J =10.5 Hz, 1H), 1.32 (m, 2H), 1.07 (s, 9H), 0.86 (s, 3H), 0.84 (s, 3H), 0.81 (t, J = 7.5 Hz, 3H) |
| 13 | 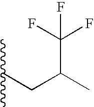 | 387.0 (M + H) | (CDCl₃) 9.32 (s, 1H), 2.91 (m, 1H), 2.85 (dd, J = 4.0 Hz, J = 15.5 Hz, 1H), 2.71 (dd, J = 5.0 Hz, J = 16.5 Hz, 1H), 2.61 (dd, J = 3.0 Hz, J = 15.5 Hz, 1H), 2.47 (q, J = 9.0 Hz, 2H), 2.38 (m, 1H), 2.00 (dd, J = 3.5 Hz, J = 12.5 Hz, 1H), 1.58 (m, 1H), 1.32 (q, J = 7.5 Hz, 2H), 1.20 (d, J = 7.0 Hz, 3H), 0.87 (s, 3H), 0.85 (s, 3H), 0.82 (t, J = 7.5 Hz, 3H) |
| 14 | 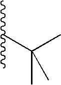 | 333.1 (M + H) | (CDCl₃) 8.33 (s, 1H), 2.71 (dd, J = 5.0 Hz, J = 16.5 Hz, 1H), 2.60 (dd, J = 5.0 Hz, J = 16.5 Hz, 1H), 2.46 (m, 1H), 2.37 (m, 1H), 1.79 (m, 1H), 1.57 (ddt, J = 2.0 Hz, J = 4.5 Hz, J = 18.5 Hz, 1H), 1.31 (m, 1H), 1.31 (s, 9H), 0.86 (s, 3H), 0.84 (s, 3H), 0.81 (t, J = 7.5 Hz, 3H) |

Using the procedures outlined in Example 8, Steps A and B, the following compounds were prepared.

### EXAMPLE 15

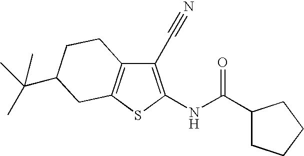

### N-(6-tert-Butyl-3-cyano-4,5,6,7-tetrahydro-1-benzothien-2-yl)cyclopentanecarboxamide

¹H NMR (500 MHz, CDCl₃) 8.54 (s, 1H), 2.84 (quint., J=8.0 Hz, 1H), 2.74 (m, 2), 2.53 (m, 1H), 2.40 (m, 1H), 2.05 (m, 1H), 1.96 (m, 1H), 1.87 (m, 1H), 1.82 (m, 1H), 1.67 (m, 1H), 1.50 (dt, J=5.0 Hz, J=12.0 Hz, 1H), 1.34 (m, 1H), 0.97 (s, 9H); mass spectrum (ES) m/e=331.3 (M+H).

### EXAMPLE 16

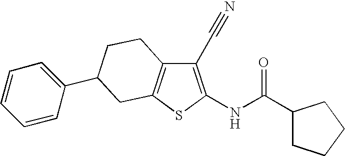

### N-(3-Cyano-6-phenyl-4,5,6,7-tetrahydro-1-benzothien-2-yl)cyclopentanecarboxamide

¹H NMR (500 MHz, CDCl₃) 8.64 (s, 1H), 7.28 (m, 3H), 7.24 (m, 2H), 3.06 (m, 1H), 2.95 (dd, J=5.5 Hz, J=16.5 Hz, 1H), 2.86 (quint., J=8.0 Hz, 1H), 2.74 (m, 2H), 2.18 (dd, J=2.5 Hz, J=11.5 Hz, 1H), 1.98 (m, 2H), 1.84 (m, 2H), 1.70 (m, 2H); mass spectrum (ES) m/e=351.2 (M+H).

### EXAMPLE 17

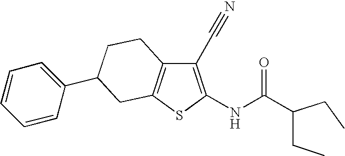

### N-(3-Cyano-6-phenyl-4,5,6,7-tetrahydro-1-benzothien-2-yl)-2-ethylbutanamide

¹H NMR (500 MHz, CDCl₃) 8.60 (s, 1H), 7.35 (m, 3H), 7.26 (m, 2H), 3.04 (m, 1H), 2.96 (dd, J=5.5 Hz, J=16.5 Hz, 1H), 2.79 (m, 1H), 2.75 (m, 1H), 2.28 (m, 1H), 2.20 (m, 1H), 1.98 (m, 1H), 1.79 (m, 2H), 1.66 (m, 2H), 0.97 (t, J=7.0 Hz, 6H); mass spectrum (ES) m/e =353.2 (M+H).

### EXAMPLE 18

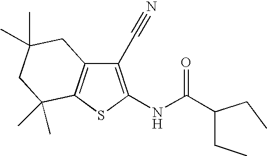

### N-(3-Cyano-5,5,7,7-tetramethyl-4,5,6,7-tetrahydro-1-benzothien-2-yl)-2-ethylbutanamide

¹H NMR (500 MHz, CDCl₃) 8.49 (s, 1H), 2.37 (s, 2H), 2.25 (m, 1H), 1.76 (m, 2H), 1.64 (m, 2H), 1.59 (s, 2H), 1.34 (s, 6H), 1.05 (s, 6H), 0.97 (t, J=7.5 Hz, 6H); mass spectrum (ES) m/e=333.2 (M+H).

### EXAMPLE 19

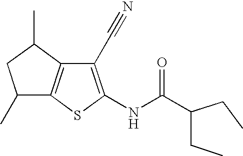

### N-(3-Cyano-4,6-dimethyl-5,6-dihydro-4H-cyclopenta[b]thien-2-yl)-2-ethylbutanamide (obtained as a 1:1 mixture of cis and trans diastereomers)

¹H NMR (500 MHz, CDCl₃) 8.79 (s, 1H); 3.40 (q, 1H), 3.36 (q, 1H), 3.24 (q, 1H); 3.19 (q, 1H), 2.85 (m, 1H), 2.33 (m, 2H), 2.26 (t, J=13.0 Hz, 2H), 1.70 (m, 4H), 1.62 m, 4H), 1.50 (m, 1H), 1.36 (d, J=7.0 Hz, 2H), 1.32 (m, 2H), 1.22 (d, 2H), 0.94 (m, 12 H); mass spectrum (ES) m/e=291.2 (M+H).

### EXAMPLE 20

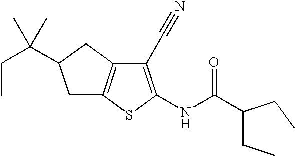

### Step A. 2-Amino-5-tert-pentyl-5,6-dihydro-4H-cyclopenta[b]thiophene-3-carbonitrile and 2-amino-6-tert-pentyl-5,6-dihydro-4H-cyclopenta[b]thiophene-3-carbonitrile

The title compound was prepared via the sequence outlined in Scheme 1. Thus, to 3-tert-pentylcyclopentanone in 10 mL of EtOH was added 2.14 g (32.5 mmol) of malononitrile, followed by 4.25 mL (48.7 mmol) of morpholine, then 2.08 g (64.9 mmol) of elemental sulfur. The mixture was stirred at ambient temperature for 16 h, then diluted with an equal volume of saturated aqueous NaHCO₃. The mixture was extracted twice with dichloromethane, and the combined organic layers were dried (Na₂SO₄) and concentrated in vacuo. Purification by flash chromatography (15% EtOAc in hexane) afforded a 4:1 mixture of the title compounds. This mixture was carried directly into Step B.

### Step B. N-(3-Cyano-5-tert-pentyl-5,6-dihydro-4H-cyclopenta[b]thien-2-yl)-2-ethylbutanamide

To the intermediate prepared in Step A in 3 mL of dichloromethane was added 0.148 mL (0.850 mmol) of di-iso-propylethylamine, followed by 0.086 mL (0.625 mmol) of 2-ethylbutanoyl chloride. After 4 h at ambient temperature, the mixture was diluted with an equal volume of saturated aqueous NaHCO₃ and extracted twice with dichloromethane. The combined organic layers were dried (Na₂SO₄) and concentrated in vacuo. Purification preparative HPLC (Chiralpak AD column, 2% EtOH in heptane) afforded the title compound as a white solid.

¹H NMR (500 MHz, CDCl₃) 8.23 (s, 1H), 2.84 (m, 1H), 2.72 (m, 1H), 2.63 (m, 1H), 2.22 (m, 1H), 1.74 (m, 2H), 1.62 (m, 2H), 1.32 (q, J=7.5 Hz, 2H), 0.96 (t, J=7.5 Hz, 3H), 0.88 (s, 6H); mass spectrum (ES) m/e=333.1 (M+H).

### EXAMPLE 21

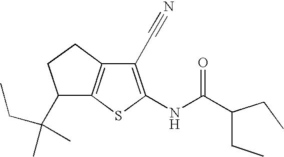

### N-(3-Cyano-6-tert-pentyl-5,6-dihydro-4H-cyclopenta[b]thien-2-yl)-2-ethylbutanamide

The title compound was obtained as a white solid byproduct of the reaction sequence outlined in Example 20.

¹H NMR (500 MHz, CDCl₃) 8.39 (s, 1H), 3.18 (t, J=8.5 Hz, 1H), 2.78 (m, 1H), 2.70 (m, 1H), 2.40 (m, 1H), 2.22 (m, 2H), 1.76 (m, 2H), 1.62 (m, 2H), 1.39 (m, 2H), 0.97 (t, J=7.0 Hz, 3H), 0.96 (t, J=7.0 Hz, 3H), 0.91 (s, 3H), 0.88 (s, 3H); mass spectrum (ES) m/e=333.1 (M+H).

### EXAMPLE 22

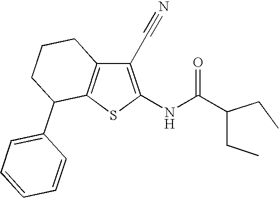

### N-(3-Cyano-7-phenyl-4,5,6,7-tetrahydro-1-benzothien-2-yl)-2-ethylbutanamide

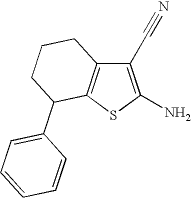

### Step A. 2-Amino-7-phenyl-4,5,6,7-tetrahydro-1-benzothiophene-3-carbonitrile

The title compound was prepared via the sequence outlined in Scheme 1. Thus, to 3-phenylcyclohexanone in 20 mL of EtOH was added 0.190 g (2.88 mmol) of malononitrile, followed by 0.252 mL (2.88 mmol) of morpholine, then 0.092 g (2.88 mmol) of elemental sulfur. The mixture was stirred at ambient temperature for 16 h, then diluted with an equal volume of saturated aqueous NaHCO₃. The mixture was extracted twice with dichloromethane, and the combined organic layers were dried (Na₂SO₄) and concentrated in vacuo. Purification by flash chromatography (15% EtOAc in hexane) afforded the title compound as a white solid.

### Step B. N-(3-Cyano-7-phenyl-4,5,6,7-tetrahydro-1-benzothien-2-yl)-2-ethylbutanamide

To the intermediate prepared in Step A in 3 mL of dichloromethane was added 0.070 mL (0.40 mmol) of di-iso-propylethylamine, followed by 0.040 mL (0.290 mmol) of 2-ethylbutanoyl chloride. After 16 h at ambient temperature, the mixture was diluted with an equal volume of saturated aqueous NaHCO₃ and extracted twice with dichloromethane. The combined organic layers were dried (Na₂SO₄) and concentrated in vacuo. Purification by flash chromatography (10% EtOAc in hexane) afforded the title compound as a white solid.

¹H NMR (500 MHz, CDCl₃) 9.46 (s, 1H), 7.31 (m, 2H), 7.19 (m, 3H), 4.03 (dd, J=6.0 Hz, J=7.5 Hz, 1H), 2.72 (m, 2H), 2.38 (m, 1H), 2.22 (m, H), 2.01 (m, 1H), 1.90 (m, 1H), 1.88 (m, 1H), 1.71 (m, 2H), 1.59 (m, 2H), 0.94 (t, J=7.0 Hz, 6H); mass spectrum (ES) m/e=353.2 (M+H).

### EXAMPLE 23

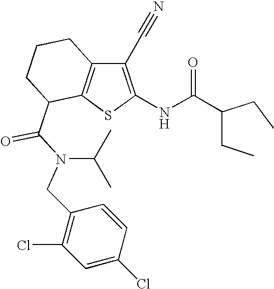

### 3-Cyano-N-(2,4-dichlorobenzyl)-2-[(2-ethylbutanoyl)amino]-N-isopropyl-4,5,6,7-tetrahydro-1-benzothiophene-7-carboxamide

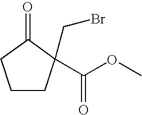

### Step A. Methyl 1-(bromomethyl)-2-oxocyclopentanecarboxylate

A solution of methyl-2-oxocyclopentanoate in 6 mL of tetrahydrofuran was added to a suspension 0.273 g (10.8 mmol) of 95% sodium hydride in 15 mL of tetrahydrofuran, followed by 1.93 g (10.8 mmol) of hexamethylphosphorylamide. After 1 h at ambient temperature, the suspension was treated with 3.15 mL (45.0 mmol) of dibromomethane, and heated to 80° C. for 10 h. The mixture was then cooled to ambient temperature and diluted with 100 mL of diethylether. The mixture was washed twice with an equal volume of H₂O, then the organic layer was dried over sodium sulfate and concentrated in vacuo. Purification by flash chromatography (20% EtOAc in hexane) afforded the title compound.

¹H NMR (500 MHz, CDCl₃) 3.76 (d, J=10.5 Hz, 1H), 3.74 (s, 3H), 3.65 (d, J=10.5 Hz, 1H), 2.58 (m, 1H), 2.50 (m, 1H), 2.46 (m, 1H), 2.30 (m, 2H), 2.10 (m, 1H), 2.03 (m, 1H).

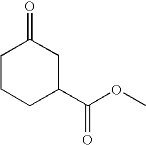

### Step B. Methyl 3-oxocyclohexanecarboxylate

To a solution of the material isolated in Step A in 80 mL of benzene was added 1.20 mL (0.427 mmol) of tri-n-butyltin hydride, followed by 0.064 g of AIBN. The mixture was heated to 85° C. for 3 h, then cooled to ambient temperature and concentrated in vacuo. Purification by flash chromatography (20% EtOAc in hexane) afforded the title compound.

¹H NMR (500 MHz, CDCl₃) 3.62 (s, 3H), 2.75 (m, 1H), 2.49 (m, 2H), 2.29 (m, 2H), 2.03 (m, 2H), 1.79 (m, 1H), 1.67 (m, 1H).

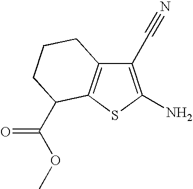

### Step C. Methyl 2-amino-3-cyano-4,5,6,7-tetrahydro-1-benzothiophene-7-carboxylate

To a solution of the title compound from Step B in 5 mL of ethyl alcohol was added 0.059 g (0.90 mmol) of malononitrile, followed by 0.118 mL (1.34 mmol) of morpholine, and 0.029 g (0.90 mmol) of elemental sulfur. After 4 h at ambient temperature, the mixture was concentrated in vacuo and purified by flash chromatography (30% EtOAc in hexane), affording a 3:1 mixture of the 5-methlcarboxylate and the 7-methylcarboxylate title compound. This mixture was carried on to the next step

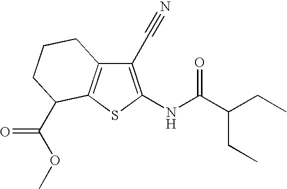

### Step D. Methyl 3-cyano-2-[(2-ethylbutanoyl)amino]-4,5,6,7-tetrahydro-1-benzothiophene-7-carboxylate

To a solution of the mixture of isomers isolated in Step C in 3 mL of dichloromethane was added 0.470 mL (3.03 mmol) of di-iso-propylethylamine, followed by 0.250 mL (2.02 mmol) of 2-ethylbutanoyl chloride. After 72 h at ambient temperature, the reaction was diluted with 50 ml of dichloromethane, followed by 50 mL of saturated aqueous NaHCO₃. The organic layer was dried (Na₂SO₄) and concentrated in vacuo. Purification by preparative thin layer chromatography (25% EtOAc in hexane) afforded the title compound isomerically pure.

¹H NMR (500 MHz, CDCl₃) 9.58 (s, 1H), 3.72 (s, 3H), 3.70 (t, J =4.0 Hz, 1H), 2.59 (m, 1H), 2.37 (m, 1H), 2.16 (m, 1H), 1.97 (m, 2H), 1.82 (m, 1H), 1.71 (m, 2H), 1.56 (m, 2H), 0.91 (t, J=7.5 Hz, 6H); mass spectrum (ES) m/e=335.2 (M+H).

### Step E. 3-Cyano-N-(2,4-dichlorobenzyl)-2-[(2-ethylbutanoyl)amino]-N-isgpropyl-4,5,6,7-tetrahydro-1-benzothiophene-7-carboxamide

To a solution of the material isolated in Step D in 1.0 mL of tetrahydrofuran was added 1.0 mL of methanol, followed by 1.0 mL of 0.1 N aqueous lithium hydroxide. After 2 h at ambient temperature, the mixture was diluted with 1.0 mL of 0.1 N aqueous HCl, and concentrated in vacuo. To a solution of 0.010 g (0.031 mmol) of this material in 1.0 mL of DMF was added 0.011 mL (0.062 mmol) of di-iso-propylethylamine, followed by 0.013 g (0.062 mmol) of N-(2,4-dichlorobenzyl)propan-2-amine and 0.018 g (0.047 mmol) of HATU. After 3 h at ambient temperature the mixture was diluted with 30 mL of dichloromethane and washed twice with an equal volume of saturated aqueous NaHCO₃. The organic layer was dried (Na—₂SO₄) and concentrated in vacuo. Purification by flash chromatography (25% EtOAc in hexane) afforded the title compound.

¹H NMR (500 MHz, CDCl₃, 1:1 mixture of rotamers) 9.00 (s, 1H), 8.78 (s, 1H), 7.39 (m, 2H), 7.27 (m, 2H), 7.21 (d, J=7.0 Hz, 1H), 7.13 (d, J=8.5 Hz, 1H), 4.95 (quint., J=7.0 Hz, 1H), 4.60 (s, 2H), 4.39 (quint., J=6.5 Hz, 1H), 4.15 (m, 1H), 3.52 (t, J=6.5 Hz, 1H), 2.65 (m, 2H), 2.18 (m, 2H), 2.07 (m, 1H), 1.97 (m, 1H), 1.72 (m, 2H), 1.60 (m, 2H), 1.29 (d, J=6.5Hz, 3H), 1.25 (d, J=7.0 Hz, 3H), 1.16 (d, J=6.5Hz, 3H), 1.13 (d, J=6.5Hz, 2H), 0.95 (m, 12 H); mass spectrum (ES) m/e=520.2 (M+H).

Biological Assays

The ability of the compounds of the present invention to inhibit the binding of glucagon can be demonstrated using the following in vitro assays.

Glucagon Receptor Binding Assay

A stable CHO (Chinese hamster ovary) cell line expressing cloned human glucagon receptor was maintained as described (Chicchi et al. J Biol Chem 272, 7765-9(1997); Cascieri et al. J Biol Chem 274, 8694-7(1999)). To determine antagonistic binding affinity of compounds 0.002 mg of cell membranes from these cells were incubated with ¹²⁵I-Glucagon (New England Nuclear, MA) in a buffer containing 50 mM Tris-HCl (pH 7.5), 5 mM MgCl₂, 2 mM EDTA, 12% Glycerol, and 0.200 mg WGA coated PVT SPA beads (Amersham), ±compounds or 0.001 mM unlabeled glucagon. After 4-12 hours incubation at room temperature, the radioactivity bound to the cell membranes was determined in a radioactive emission detection counter (Microbeta-Wallace). Data was analyzed using the software program Prism® from GraphPad. The IC₅₀ were calculated using non-linear regression analysis assuming single site competition.

High Throughput Screening (HTS) Protocol for Glucagon Receptor Binding Assay

Another form of the binding assay was developed suitable for high-throughput screening for modulators of receptor activity. Fully automated or semi-automated protocols and robotic and workstation instruments were utilized for the HTS assay as would be recognized by those practiced in the art. In a typical configuration of the assay, 0.002 mg of cell membrane (as described above) were preincubated with 0.200 mg of WGA-coated PVT beads in buffer containing 100 mM Tris-HCl pH 7.5, 10 mM MgCl₂, 4 mM EDTA, 24% Glycerol, and 0.2% BSA. The membrane/bead mixture was then dispensed (0.050 mL) into each well of 96-well plates (Wallac Isoplates, white clear bottom) containing 0.100 mL of test compounds or control solutions. A second addition (0.050 mL) was then dispensed into the wells of the plate containing ¹²⁵I-Glucagon (approximately 25,000 CPM). The solutions were dispensed using a Multidrop Stacker 20 (Titertek) liquid dispenser. An adhesive plate seal (Packard) was applied and the plates were shaken for 5 minutes. The plates were further incubated at ambient temperature for several hours for establishment of equilibrium (typically 5 hours) and the signal was stable for up to three days. The plates were read in a scintillation counter (Wallac Microbeta) for 1 min/well. Activity of test compounds was calculated by comparing to the total scintillation signal (CPM) of control samples with no compound and with 0.001 mM unlabeled-glucagon.

Inhibition of Glucagon-Stimulated Intracellular cAMP Formation

Exponentially growing CHO cells expressing human glucagon receptor were harvested with the aid of enzyme-free dissociation media (Specialty Media), pelleted at low speed, and re-suspended in cell suspension buffer [75 mM Tris-HCl pH7.5, 250 mM Sucrose, 25 mM MgCl₂, 1.5 mM EDTA, 0.1 mM Ro-20-1724 (Biomol, Inc.), 0.2% bovine serum albumin and one tablet of complete™ (Boehringer), which contains a cocktail of protease inhibitors, for each 50 ml of buffer]. An adenylate cyclase assay was setup using an Adenylate Cyclase Assay kit (SMP-004B) from New England Nuclear (NEN) as per manufacturer instructions. Briefly, compounds were diluted from stocks in a cell stimulation buffer supplied with the kit. Cells prepared as above were preincubated in flash plates coated with anti-cAMP antibodies (NEN) in presence of compounds or DMSO controls for 40 minutes, and then stimulated with glucagon (250 pM) for an additional 40 minutes. The cell stimulation was stopped by addition of equal amount of a detection buffer containing lysis buffer as well as ¹²⁵I-labeled cAMP tracer (NEN). After 3-6 h of incubation at room temperature the bound radioactivity was determined in a liquid scintillation counter (TopCount-Packard Instruments). Activity of test compounds was calculated by comparing to the total scintillation signal (CPM) of control samples with no compound and with 0.001 mM unlabeled-glucagon.

Certain embodiments of the invention has been described in detail; however, numerous other embodiments are contemplated as falling within the invention. Thus, the claims are not limited to the specific embodiments described herein. All patents, patent applications and publications that are cited herein are hereby incorporated by reference in their entirety.

1. A method of treating type 2 diabetes mellitus in a mammalian patient in need of such treatment, which comprises administering to said patient an anti-diabetic effective amount of a compound represented by formula I: 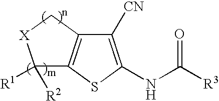
or a pharmaceutically acceptable salt or solvate thereof wherein:
X is CR⁵R⁶;
at least one of R¹, R², R⁵ and R⁶ is present that is other than H;
R¹ is selected from the group consisting of: H, C₁₋₁₀alkyl, C₃₋₇cycloalkyl and Aryl, said alkyl, cycloalkyl and Aryl being optionally substituted with 1-4 substituents independently selected from R^{13;}
R² is selected from the group consisting of: R¹ as defined above, —C(O)₂R⁷ and —CONR⁷R⁸;
m and n are selected from 0, 1, 2 and 3, such that the sum of m and n is 2 or 3, and when m is greater than 1, no more than one R¹ and no more than one R² can be other than H;
R³ is selected from the group consisting of: C₁₋₁₀alkyl, C₃₋₇cycloalkyl and Aryl, said alkyl, cycloalkyl and Aryl being optionally substituted with 1-4 substituents selected from R¹³, such that when R³ represents C₁₋₁₀alkyl substituted with one R¹³ group, and R¹³ represents halo, R¹, R², R⁵ and R⁶ do not represent C₁₋₃alkyl;
R⁵ is selected from the group consisting of: H, C₁₋₁₀alkyl, C₃₋₇cycloalkyl and Aryl, said alkyl, cycloalkyl and Aryl being optionally substituted with 1-4 substituents selected from R¹³;
R⁶ is selected from the group consisting of: R¹ as defined above, HAR, Hetcy, and OR¹¹, wherein said HAR and Hetcy being optionally substituted with 1-4 substituents selected from R¹³,
or R⁵ and R⁶ can be taken in combination with the carbon atom to which they are attached and represent —O—(CH₂)₁₋₂—O— or —C(O)—;
R⁷, R¹⁰ and R¹¹ are selected from the group consisting of: R¹ as defined above, HAR and Hetcy, said HAR and Hetcy being optionally substituted with 1-4 substituents selected from R¹³;
R⁸, R⁹ and R¹² are selected from the group consisting of: C₁₋₁₀alkyl, C₃₋₇cycloalkyl, Aryl, HAR and Hetcy, said alkyl, cycloalkyl, Aryl, HAR and Hetcy being optionally substituted with 1-4 substituents selected from R¹³;
or alternatively, R⁷, R⁸, R⁹ and R¹⁰ are as defined above, and R¹¹ and R¹² are taken together with the atoms to which they are attached and form a 5-8 membered ring optionally containing 1-2 heteroatoms selected from O, S and N, and optionally substituted with 1-4 substituents selected from R¹³;
each R¹³ is selected from the group consisting of: halo, NR¹⁴R¹⁵, C₁₋₄alkyl, C₃₋₇₋cycloalkyl, Aryl, HAR, Hetcy, CF₃, OCF3, OR¹⁵, NO₂, S(O)ₓR¹⁴, SR¹⁴, S(O)ₓNR¹⁴R¹⁵, O(CR¹⁶R¹⁷)_{y}NR¹⁴R¹⁵, C(O)R¹⁴, CO₂R¹⁵, CO₂(CR¹⁶R¹⁷)_{y}CONR¹⁴R¹⁵, OC(O)R¹⁴, CN, C(O)NR¹⁴R¹⁵, NR¹⁵C(O)R¹⁴, NR¹⁵C(O)OR¹⁴, NR¹⁵C(O)NR¹⁶R¹⁴ and CR¹⁵(N—OR¹⁴),
wherein x is 1 or 2, and y is an integer from 1-4,
said alkyl, cycloalkyl, Aryl, HAR and Hetcy being optionally substituted with 1-4 substituents selected from R¹⁸;
R¹⁴, R¹⁵, R¹⁶ and R¹⁷ are independently selected from the group consisting of: H, C₁₋₁₀alkyl, C₃₋₇cycloalkyl, Aryl and Ar—C₁₋₁₀alkyl;
and each R¹⁸ is independently selected from the group consisting of: halogen, CN, C₁₋₄alkyl, OH, CF₃, Aryl, Aryloxy, CO₂H and CO₂C₁₋₄ alkyl, said Aryl and the Aryl portion of Aryloxy being optionally substituted with up to 4 halo groups, and up to 2 C₁₋₄ alkyl, OH, CF₃ or CN groups.

2. A method of treating type 2 diabetes mellitus in accordance with claim 1 wherein R¹ is selected from the group consisting of: H, C₁₋₆alkyl and C₃₋₆cycloalkyl, said alkyl and cycloalkyl being optionally substituted with 1-3 substituents independently selected from R¹³.

3. A method of treating type 2 diabetes mellitus in accordance with claim 1 wherein R² is selected from the group consisting of: H, C₁₋₆alkyl, C₃₋₆cycloalkyl, Aryl and C(O)NR⁷R⁸, said alkyl, cycloalkyl and Aryl groups being optionally substituted with 1-3 substituents independently selected from R¹³;
R⁷ is selected from the group consisting of: H and C₁₋₆ alkyl, optionally substituted with 1-3 R¹³ groups;
R⁸ is selected from the group consisting of: C₁₋₆ alkyl, C₃₋₆ cycloalkyl, and Aryl, optionally substituted with 1-3 R¹³ groups;
each R¹³ is independently selected from the group consisting of: halo, Aryl, CF₃ and OCF₃, and Aryl is optionally substituted with 1-3 R¹⁸ groups, which are each independently selected from halo, CH₃, OH, CF₃ and CO₂H.

4. A method of treating type 2 diabetes mellitus in accordance with claim 1 wherein R³ is selected from the group consisting of: C₁₋₁₀alkyl and C₃₋₇cycloalkyl, said alkyl and cycloalkyl being optionally substituted with 1-3 substituents selected from R¹³, such that when R³represents C₁₋₁₀ alkyl substituted with one R¹³ group, and R¹³ represents halo, R¹, R², R⁵ and R⁶ do not represent C₁₋₃alkyl.

5. A method of treating type 2 diabetes mellitus in accordance with claim 1 wherein R⁵ is selected from the group consisting of: H, C₁₋₆alkyl and C₃₋₆cycloalkyl, said alkyl and cycloalkyl being optionally substituted with 1-3 substituents selected from R¹³.

6. A method of treating type 2 diabetes mellitus in accordance with claim 1 wherein R⁶ is selected from the group consisting of: H, C₁₋₆alkyl and C₃₋₆cycloalkyl, said alkyl and cycloalkyl being optionally substituted with 1-3 substituents selected from R¹³.

7. A method of treating type 2 diabetes mellitus in accordance with claim 1 wherein each R¹³ is selected from the group consisting of: halo, C₁₋₄alkyl, C₃₋₆cycloalkyl, Aryl, CF₃ and OCF₃, and Aryl is optionally substituted with 1-3 R¹⁸ groups, which are independently selected from halo, CH₃, OH, CF₃ and CO₂H.

8. A method of treating type 2 diabetes mellitus in accordance with claim 1 wherein:
R¹ is selected from the group consisting of: H, C₁₋₆alkyl and C₃₋₆cycloalkyl, said alkyl and cycloalkyl being optionally substituted with 1-3 substituents independently selected from R¹³;
R² is selected from the group consisting of: H, C₁₋₆alkyl, C₃₋₆cycloalkyl, Aryl and C(O)NR⁷R⁸, said alkyl, cycloalkyl and Aryl groups being optionally substituted with 1-3 substituents independently selected from R¹³;
R⁷ is selected from the group consisting of: H and C₁₋₆ alkyl, optionally substituted with 1-3 R¹³ groups;
R⁸ is selected from the group consisting of: C₁₋₆ alkyl, C₃₋₆ cycloalkyl, and Aryl, optionally substituted with 1-3 R¹³ groups;
each R¹³ is independently selected from the group consisting of: halo, Aryl, CF₃ and OCF₃, and Aryl is optionally substituted with 1-3 R¹⁸ groups, which are each independently selected from halo, CH₃, OH, CF₃ and CO₂H;
R³ is selected from the group consisting of: C₁₋₁₀alkyl and C₃₋₇cycloalkyl, said alkyl and cycloalkyl being optionally substituted with 1-3 substituents selected from R¹³, such that when R³represents C₁₋₁₀ alkyl substituted with one R¹³ group, and R¹³ represents halo, R¹, R², R⁵ and R⁶ do not represent C₁₋₃alkyl;
R⁵ is selected from the group consisting of: H, C₁₋₆alkyl and C₃₋₆cycloalkyl, said alkyl and cycloalkyl being optionally substituted with 1-3 substituents selected from R¹³;
R⁶ is selected from the group consisting of: H, C₁₋₆alkyl and C₃₋₆cycloalkyl, said alkyl and cycloalkyl being optionally substituted with 1-3 substituents selected from R¹³, and
each R¹³ is selected from the group consisting of: halo, C₁₋₄alkyl, C₃₋₆cycloalkyl, Aryl, CF₃ and OCF₃, and Aryl is optionally substituted with 1-3 R¹⁸ groups, which are independently selected from halo, CH₃, OH, CF₃ and CO₂H.

9. A method of treating type 2 diabetes mellitus in accordance with claim 1 wherein the compound administered is selected from the group consisting of: N-(3-cyano-6-methyl-4,5,6,7-tetrahydro-1-benzothien-2-yl)-2-ethylbutanamide; N-(3-cyano-6-tert-pentyl-4,5,6,7-tetrahydro-1-benzothien-2-yl)-2-ethylbutanamide; N-(3-cyano-6-tert-pentyl-4,5,6,7-tetrahydro-1-benzothien-2-yl)-3-methylbutanamide; N-(6-tert-butyl-3-cyano-4,5,6,7-tetrahydro-1-benzothien-2-yl)decanamide; N-(3-cyano-6-tert-pentyl-4,5,6,7-tetrahydro-1-benzothien-2-yl)bicyclo[2.2.1]heptane-2-carboxamide; N-(3-cyano-6-ethyl-4,5,6,7-tetrahydro-1-benzothien-2-yl)-2-phenylcyclopropanecarboxamide; N-(3-cyano-6-tert-pentyl-4,5,6,7-tetrahydro-1-benzothien-2-yl)-2-phenylcyclopropanecarboxamide; N-(3-cyano-6-tert-pentyl-4,5,6,7-tetrahydro-1-benzothien-2-yl)cyclopentanecarboxamide; N-(3-cyano-6-tert-pentyl-4,5,6,7-tetrahydro-1-benzothien-2-yl)-2,2,3,3-tetramethylcyclopropanecarboxamide; N-(3-cyano-6-tert-pentyl-4,5,6,7-tetrahydro-1-benzothien-2-yl)-3-cyclohexylpropanamide; N-(3-cyano-6-tert-pentyl-4,5,6,7-tetrahydro-1-benzothien-2-yl)-3-phenylpropanamide; N-(3-cyano-6-tert-pentyl-4,5,6,7-tetrahydro-1-benzothien-2-yl)-3,3-dimethylbutanamide; N-(3-cyano-6-tert-pentyl-4,5,6,7-tetrahydro-1-benzothien-2-yl)-4,4,4-trifluoro-3-methylbutanamide; N-(3-cyano-6-tert-pentyl-4,5,6,7-tetrahydro-1-benzothien-2-yl)-2,2-dimethylpropanamide; N-(6-tert-butyl-3-cyano-4,5,6,7-tetrahydro-1-benzothien-2-yl)cyclopentanecarboxamide; N-(3-cyano-6-phenyl-4,5,6,7-tetrahydro-1-benzothien-2-yl)cyclopentanecarboxamide; N-(3-cyano-6-phenyl-4,5,6,7-tetrahydro-1-benzothien-2-yl)-2-ethylbutanamide; N-(3-cyano-5,5,7,7-tetramethyl-4,5,6,7-tetrahydro-1-benzothien-2-yl)-2-ethylbutanamide; N-(3-cyano-5-tert-pentyl-5,6-dihydro-4H-cyclopenta[b]thien-2-yl)-2-ethylbutanamide; N-(3-cyano-6-tert-pentyl-5,6-dihydro-4H-cyclopenta[b]thien-2-yl)-2-ethylbutanamide; N-(3-cyano-4,6-dimethyl-5,6-dihydro-4H-cyclopenta[b]thien-2-yl)-2-ethylbutanamide; N-(3-cyano-7-phenyl-4,5,6,7-tetrahydro-1-benzothien-2-yl)-2-ethylbutanamide; and 3-cyano-N-(2,4-dichlorobenzyl)-2-[(2-ethylbutanoyl)amino]-N-isopropyl-4,5,6,7-tetrahydro-1-benzothiophene-7-carboxamide.

10. A pharmaceutical composition comprised of a compound represented by formula I: 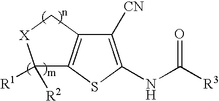
or a pharmaceutically acceptable salt or solvate thereof in combination with a pharmaceutically acceptable carrier, wherein:
X is CR⁵R⁶;
at least one of R¹, R², R⁵ and R⁶ is present that is other than H;
R¹ is selected from the group consisting of: H, C₁₋₁₀alkyl, C₃₋₇cycloalkyl and Aryl, said alkyl, cycloalkyl and Aryl being optionally substituted with 1-4 substituents independently selected from R¹³;
R² is selected from the group consisting of: R¹ as defined above, —C(O)₂R⁷ and —CONR⁷R⁸;
m and n are selected from 0, 1, 2 and 3, such that the sum of m and n is 2 or 3, and when m is greater than 1, no more than one R¹ and no more than one R² can be other than H;
R³ is selected from the group consisting of: C₁₋₁₀alkyl, C₃₋₇cycloalkyl and Aryl, said alkyl, cycloalkyl and Aryl being optionally substituted with 1-4 substituents selected from R¹³, such that when R³ represents C₁₋₁₀alkyl substituted with one R¹³ group, and R¹³ represents halo, R¹, R², R⁵ and R⁶ do not represent C₁₋₃alkyl;
R⁵ is selected from the group consisting of: H, C₁₋₁₀alkyl, C₃₋₇cycloalkyl and Aryl, said alkyl, cycloalkyl and Aryl being optionally substituted with 1-4 substituents selected from R¹³;
R⁶ is selected from the group consisting of: R¹ as defined above, HAR, Hetcy, and OR¹¹, wherein said HAR and Hetcy being optionally substituted with 1-4 substituents selected from R¹³,
or R⁵ and R⁶ can be taken in combination with the carbon atom to which they are attached and represent —O—(CH₂)₁₋₂—O— or —C(O)—;
R⁷, R¹⁰ and R¹¹ are selected from the group consisting of: R¹ as defined above, HAR and Hetcy, said HAR and Hetcy being optionally substituted with 1-4 substituents selected from R¹³;
R⁸, R¹⁰ and R¹² are selected from the group consisting of: C₁₋₁₀alkyl, C₃₋₇cycloalkyl, Aryl, HAR and Hetcy, said alkyl, cycloalkyl, Aryl, HAR and Hetcy being optionally substituted with 1-4 substituents selected from R¹³;
or alternatively, R⁷, R⁸, R⁹ and R¹⁰ are as defined above, and R¹¹ and R¹² are taken together with the atoms to which they are attached and form a 5-8 membered ring optionally containing 1-2 heteroatoms selected from O, S and N, and optionally substituted with 1-4 substituents selected from R¹³;
each R¹³ is selected from the group consisting of: halo, NR¹⁴R¹⁵, C₁₋₄alkyl, C₃₋₇₋cycloalkyl, Aryl, HAR, Hetcy, CF₃, OCF₃, OR¹⁵, NO₂, S(O)ₓR¹⁴, SR¹⁴, S(O)ₓNR¹⁴R¹⁵, O(CR¹⁶R¹⁷)_{y}NR¹⁴R¹⁵, C(O)R¹⁴, CO₂R¹⁵, CO₂(CR¹⁶R¹⁷)_{y}CONR¹⁴R¹⁵, OC(O)R¹⁴, CN, C(O)NR¹⁴R¹⁵, NR¹⁵C(O)R¹⁴, NR¹⁵C(O)OR¹⁴, NR¹⁵C(O)NR¹⁶R¹⁴ and CR¹⁵(N—OR¹⁴),
wherein x is 1 or 2, and y is an integer from 1-4,
said alkyl, cycloalkyl, Aryl, HAR and Hetcy being optionally substituted with 1-4 substituents selected from R¹⁸;
R¹⁴, R¹⁵, R¹⁶ and R¹⁷ are independently selected from the group consisting of: H, C₁₋₁₀alkyl, C₃₋₇cycloalkyl, Aryl and Ar—C₁₋₁₀alkyl;
and each R¹⁸ is independently selected from the group consisting of: halogen, CN, C₁₋₄alkyl, OH, CF₃, Aryl, Aryloxy, CO₂H and CO₂C₁₋₄ alkyl, said Aryl and the Aryl portion of Aryloxy being optionally substituted with up to 4 halo groups, and up to 2 C₁₋₄ alkyl, OH, CF₃ or CN groups,
in combination with a pharmaceutically acceptable carrier.

11. A pharmaceutical composition in accordance with claim 10 wherein: R¹ is selected from the group consisting of: H, C₁₋₆alkyl and C₃₋₆cycloalkyl, said alkyl and cycloalkyl being optionally substituted with 1-3 substituents independently selected from R¹³.

12. A pharmaceutical composition in accordance with claim 10 wherein:
R² is selected from the group consisting of: H, C₁₋₆alkyl, C₃₋₆cycloalkyl, Aryl and C(O)NR⁷R⁸, said alkyl, cycloalkyl and Aryl groups being optionally substituted with 1-3 substituents independently selected from R¹³; R⁷ is selected from the group consisting of: H and C₁₋₆ alkyl, optionally substituted with 1-3 R¹³ groups;
R⁸ is selected from the group consisting of: C₁₋₆ alkyl, C₃₋₆ cycloalkyl, and Aryl, optionally substituted with 1-3 R¹³ groups;
each R¹³ is independently selected from the group consisting of: halo, Aryl, CF₃ and OCF₃, and Aryl is optionally substituted with 1-3 R¹⁸ groups, which are each independently selected from halo, CH₃, OH, CF₃ and CO₂H.

13. A pharmaceutical composition in accordance with claim 10 wherein R³ is selected from the group consisting of: C₁₋₁₀alkyl and C₃₋₇cycloalkyl, said alkyl and cycloalkyl being optionally substituted with 1-3 substituents selected from R¹³, such that when R³represents C₁₋₁₀alkyl substituted with one R¹³ group, and R¹³ represents halo, R¹, R², R⁵ and R⁶ do not represent C₁₋₃alkyl.

14. A pharmaceutical composition in accordance with claim 10 wherein R⁵ is selected from the group consisting of: H, C₁₋₆alkyl and C₃₋₆cycloalkyl, said alkyl and cycloalkyl being optionally substituted with 1-3 substituents selected from R¹³.

15. A pharmaceutical composition in accordance with claim 10 wherein R⁶ is selected from the group consisting of: H, C₁₋₆alkyl and C₃₋₆cycloalkyl, said alkyl and cycloalkyl being optionally substituted with 1-3 substituents selected from R¹³.

16. A pharmaceutical composition in accordance with claim 10 wherein each R¹³ is selected from the group consisting of: halo, C₁₋₄alkyl, C₃₋₆cycloalkyl, Aryl, CF₃ and OCF₃, and Aryl is optionally substituted with 1-3 R¹⁸ groups, which are independently selected from halo, CH₃, OH, CF₃ and CO₂H.

17. A pharmaceutical composition in accordance with claim 10 wherein:
R¹ is selected from the group consisting of: H, C₁₋₆alkyl and C₃₋₆cycloalkyl, said alkyl and cycloalkyl being optionally substituted with 1-3 substituents independently selected from R¹³;
R² is selected from the group consisting of: H, C₁₋₆alkyl, C₃₋₆cycloalkyl, Aryl and C(O)NR⁷R⁸, said alkyl, cycloalkyl and Aryl groups being optionally substituted with 1-3 substituents independently selected from R¹³;
R⁷ is selected from the group consisting of: H and C₁₋₆ alkyl, optionally substituted with 1-3 R¹³ groups;
R⁸ is selected from the group consisting of: C₁₋₆ alkyl, C₃₋₆ cycloalkyl, and Aryl, optionally substituted with 1-3 R¹³ groups;
each R¹³ is independently selected from the group consisting of: halo, Aryl, CF₃ and OCF₃, and Aryl is optionally substituted with 1-3 R¹⁸ groups, which are each independently selected from halo, CH₃, OH, CF₃ and CO₂H;
R³ is selected from the group consisting of: C₁₋₁₀alkyl and C₃₋₇cycloalkyl, said alkyl and cycloalkyl being optionally substituted with 1-3 substituents selected from R¹³, such that when R³ represents C₁₋₁₀alkyl substituted with one R¹³ group, and R¹³ represents halo, R¹, R², R⁵ and R⁶ do not represent C¹³alkyl;
R⁵ is selected from the group consisting of: H, C₁₋₆alkyl and C₃₋₆cycloalkyl, said alkyl and cycloalkyl being optionally substituted with 1-3 substituents selected from R¹³;
R⁶ is selected from the group consisting of: H, C₁₋₆alkyl and C₃₋₆cycloalkyl, said alkyl and cycloalkyl being optionally substituted with 1-3 substituents selected from R¹³, and
each R¹³ is selected from the group consisting of: halo, C₁₋₄alkyl, C₃₋₆cycloalkyl, Aryl, CF₃ and OCF₃, and Aryl is optionally substituted with 1-3 R¹⁸ groups, which are independently selected from halo, CH₃, OH, CF₃ and CO₂H. Within this aspect of the invention, all other variables are as originally defined.

18. A pharmaceutical composition in accordance with claim 10 wherein the compound of formula I is selected from the group consisting of: N-(3-cyano-6-methyl-4,5,6,7-tetrahydro-1-benzothien-2-yl)-2-ethylbutanamide; N-(3-cyano-6-tert-pentyl-4,5,6,7-tetrahydro-1-benzothien-2-yl)-2-ethylbutanamide; N-(3-cyano-6-tert-pentyl-4,5,6,7-tetrahydro-1-benzothien-2-yl)-3-methylbutanamide; N-(6-tert-butyl-3-cyano-4,5,6,7-tetrahydro-1-benzothien-2-yl)decanamide; N-(3-cyano-6-tert-pentyl-4,5,6,7-tetrahydro-1-benzothien-2-yl)bicyclo[2.2.1]heptane-2-carboxamide; N-(3-cyano-6-ethyl-4,5,6,7-tetrahydro-1-benzothien-2-yl)-2-phenylcyclopropanecarboxamide; N-(3-cyano-6-tert-pentyl-4,5,6,7-tetrahydro-1-benzothien-2-yl)-2-phenylcyclopropanecarboxamide; N-(3-cyano-6-tert-pentyl-4,5,6,7-tetrahydro-1-benzothien-2-yl)cyclopentanecarboxamide; N-(3-cyano-6-tert-pentyl-4,5,6,7-tetrahydro-1-benzothien-2-yl)-2,2,3,3-tetramethylcyclopropanecarboxamide; N-(3-cyano-6-tert-pentyl-4,5,6,7-tetrahydro-1-benzothien-2-yl)-3-cyclohexylpropanamide; N-(3-cyano-6-tert-pentyl-4,5,6,7-tetrahydro-1-benzothien-2-yl)-3-phenylpropanamide; N-(3-cyano-6-tert-pentyl-4,5,6,7-tetrahydro-1-benzothien-2-yl)-3,3-dimethylbutanamide; N-(3-cyano-6-tert-pentyl-4,5,6,7-tetrahydro-1-benzothien-2-yl)-4,4,4-trifluoro-3-methylbutanamide; N-(3-cyano-6-tert-pentyl-4,5,6,7-tetrahydro-1-benzothien-2-yl)-2,2-dimethylpropanamide; N-(6-tert-butyl-3-cyano-4,5,6,7-tetrahydro-1-benzothien-2-yl)cyclopentanecarboxamide; N-(3-cyano-6-phenyl-4,5,6,7-tetrahydro-1-benzothien-2-yl)cyclopentanecarboxamide; N-(3-cyano-6-phenyl-4,5,6,7-tetrahydro-1-benzothien-2-yl)-2-ethylbutanamide; N-(3-cyano-5,5,7,7-tetramethyl-4,5,6,7-tetrahydro-1-benzothien-2-yl)-2-ethylbutanamide; N-(3-cyano-5-tert-pentyl-5,6-dihydro-4H-cyclopenta[b]thien-2-yl)-2-ethylbutanamide; N-(3-cyano-6-tert-pentyl-5,6-dihydro-4H-cyclopenta[b]thien-2-yl)-2-ethylbutanamide; N-(3-cyano-4,6-dimethyl-5,6-dihydro-4H-cyclopenta[b]thien-2-yl)-2-ethylbutanamide; N-(3-cyano-7-phenyl-4,5,6,7-tetrahydro-1-benzothien-2-yl)-2-ethylbutanamide; and 3-cyano-N-(2,4-dichlorobenzyl)-2-[(2-ethylbutanoyl)amino]-N-isopropyl-4,5,6,7-tetrahydro-1-benzothiophene-7-carboxamide.

19.-20. (canceled)

21. A compound selected from the group consisting of: N-(3-cyano-6-tert-pentyl-4,5,6,7-tetrahydro-1-benzothien-2-yl)cyclopentanecarboxamide; N-(3-cyano-6-tert-pentyl-4,5,6,7-tetrahydro-1-benzothien-2-yl)-2,2,3,3-tetramethylcyclopropanecarboxamide; N-(3-cyano-6-tert-pentyl-4,5,6,7-tetrahydro-1-benzothien-2-yl)-3-cyclohexylpropanamide; N-(3-cyano-6-tert-pentyl-4,5,6,7-tetrahydro-1-benzothien-2-yl)-3-phenylpropanamide; N-(3-cyano-6-tert-pentyl-4,5,6,7-tetrahydro-1-benzothien-2-yl)-3,3-dimethylbutanamide; N-(3-cyano-6-tert-pentyl-4,5,6,7-tetrahydro-1-benzothien-2-yl)-4,4,4-trifluoro-3-methylbutanamide; N-(3-cyano-6-tert-pentyl-4,5,6,7-tetrahydro-1-benzothien-2-yl)-2,2-dimethylpropanamide; N-(6-tert-butyl-3-cyano-4,5,6,7-tetrahydro-1-benzothien-2-yl)cyclopentanecarboxamide; N-(3-cyano-6-phenyl-4,5,6,7-tetrahydro-1-benzothien-2-yl)cyclopentanecarboxamide; N-(3-cyano-6-phenyl-4,5,6,7-tetrahydro-1-benzothien-2-yl)-2-ethylbutanamide; N-(3-cyano-5,5,7,7-tetramethyl-4,5,6,7-tetrahydro-1-benzothien-2-yl)-2-ethylbutanamide; N-(3-cyano-5-tert-pentyl-5,6-dihydro-4H-cyclopenta[b]thien-2-yl)-2-ethylbutanamide; N-(3-cyano-6-tert-pentyl-5,6-dihydro-4H-cyclopenta[b]thien-2-yl)-2-ethylbutanamide; N-(3-cyano-4,6-dimethyl-5,6-dihydro-4H-cyclopenta[b]thien-2-yl)-2-ethylbutanamide; N-(3-cyano-7-phenyl-4,5,6,7-tetrahydro-1-benzothien-2-yl)-2-ethylbutanamide; and 3-cyano-N-(2,4-dichlorobenzyl)-2-[(2-ethylbutanoyl)amino]-N-isopropyl-4,5,6,7-tetrahydro-1-benzothiophene-7-carboxamide, or a pharmaceutically acceptable salt or solvate thereof.
